(19) United States
(12) Patent Application Publication
(10) Pub. No.: US 2026/0284278 A1
(43) Pub. Date: Sep. 24, 2026

(54) MEDICAL DEVICE METAL ALLOY

(71) Applicant: MiRus LLC, Marietta, GA (US)

(72) Inventors: Noah Roth, Marietta, GA (US); Jordan Bauman, Smyrna, GA (US); Ravi Enneti, Marietta, GA (US); Jay Yadav, Atlanta, GA (US)

(21) Appl. No.: 19/646,673

(22) Filed: Apr. 14, 2026

Related U.S. Application Data
(63) Continuation of application No. 18/126,070, filed on Mar. 24, 2023.
Application No. 18/126,070 is a continuation-in-part of application No. 17/586,270, filed on Jan. 27, 2022.
Application No. 18/126,070 is a continuation-in-part of application No. 18/116,677, filed on Mar. 2, 2023.
(60) Provisional application No. 63/226,270, filed on Jul. 28, 2021.
(60) Provisional application No. 63/316,077, filed on Mar. 3, 2022.

(51) Int. Cl. A61L 31/02; A61L 31/16; C22C 14/00; C22C 16/00; C22C 19/07; C22C 27/02; C22C 27/04; C22C 27/06; C22C 30/00; C22C 38/08; C22C 38/40

(52) U.S. Cl. CPC A61L 31/022; A61L 31/16; C22C 14/00; C22C 16/00; C22C 19/07; C22C 27/02; C22C 27/04; C22C 27/06; C22C 30/00; C22C 38/08; C22C 38/40

(57) ABSTRACT

A medical device that is partially or fully formed of a metal alloy; the metal alloy includes one of a) metal alloy that includes at least 15 awt % rhenium, b) at least 60 wt. % tungsten, at least 15 awt % rhenium, and at least 1 wt % molybdenum, c) at least 50 wt. % rhenium, at least 20 wt. % chromium, and 0.1-80 wt. % of an additive, d) greater than 50 wt. % titanium, 15-45 wt. % niobium, 1-10 wt. % zirconium, and 1-15 wt. % tantalum, e) greater than 50 wt. % titanium, 15-45 wt. % niobium, and 1-10 wt. %, f) 30-60 wt. % cobalt, 10-30 wt. % chromium, 5-20 wt. % iron, 5-22 wt. % nickel, and 2-12 wt. % molybdenum, g) 40-60 wt. % zirconium, and 40-60 wt. % molybdenum, h) 90-99.5 wt. % niobium, and 0.5-10 wt. % zirconium, or i) 55-75 wt. % niobium, 18-40 wt. % tantalum, 1-7 wt. % tungsten, and 0.5-4 wt. % zirconium.

The present disclosure is continuation of U.S. patent application Ser. No. 18/126,070 filed Mar. 24, 2023, which in turn is a continuation in part of U.S. patent application Ser. No. 17/586,270 filed Jan. 27, 2022, which in turn claims priority on U.S. Provisional Application Ser. No. 63/226,270 filed Jul. 28, 2021, which are both incorporated herein by reference.

The present disclosure is continuation of U.S. patent application Ser. No. 18/126,070 filed Mar. 24, 2023, which in turn is a continuation in part of U.S. patent application Ser. No. 18/116,677 filed Mar. 2, 2023, which in turn claims priority on U.S. Provisional Application Ser. No. 63/316,077 filed Mar. 3, 2022, which is incorporated herein by reference.

The disclosure relates generally to medical devices and medical device applications, and more particularly to a medical device that is at least partially formed of a biocompatible metal alloy.

### BACKGROUND OF DISCLOSURE

Stainless steel, cobalt-chromium alloys, and TiAlV alloys are some of the more common metal alloys used for medical devices. Although these alloys have been successful in forming a variety of medical devices, these alloys have several deficiencies. Refractory metal alloys and alloys that include rhenium have also been used to partially or fully form the medical device.

Many cardiovascular devices such as stents, expandable heart valves, and the like are inserted into a patient via the vascular system of a patient and then expanded at the treatment site. These devices are typically crimped onto a catheter prior to insertion into a patient. The minimum diameter to which the cardiovascular device can be crimped onto the catheter will set a limit to the size of the cardiovascular passageway (e.g., blood vessel) to which the cardiovascular device can be inserted. Smaller crimp diameters can result in reduced damage to a blood vessel and/or organ (e.g., heart, etc.) when inserting into and/or placing the cardiovascular device at the treatment site. Smaller crimp diameters can also allow the cardiovascular device to be placed in smaller diameter blood vessels (e.g., blood vessels located in the brain, etc.).

The crimp diameter of the expandable cardiovascular device can be reduced by reducing the thickness and/or size of the frame, struts, etc., of the cardiovascular device. However, such reduction in size also affects the strength of the cardiovascular device after being expanded. After the cardiovascular device is expanded, it must retain its expanded shape at the treatment area, otherwise the cardiovascular device could become dislodged from the treatment area, could damage the treatment area, and/or fail to properly function at the treatment area. As such, cardiovascular devices formed of tradition materials such as stainless steel [e.g., 316L: 17-19 wt. % chromium, 13-15 wt. % nickel, 2-4 wt. % molybdenum, 2 wt. % max manganese, 0.75 wt. % max silicon, 0.03 wt. % max carbon, balance iron] and cobalt-chromium alloys (e.g., MP35N: 19-21 wt. % chromium, 34-36 wt. % nickel, 9-11 wt. % molybdenum, 1 wt. % max iron, 1 wt. % max titanium, 0.15 wt. % max manganese, 0.15 wt. % max silver, 0.025 wt. % max carbon, balance cobalt) are required to maintain a frame and/or strut size/thickness that limits how small of crimping diameter can be obtained by the crimped cardiovascular device. Other types of cobalt-chromium alloys that have been used are Phynox and Elgiloy alloy (38-42 wt. % cobalt, 18-22 wt. % chromium, 14-18 wt. % iron, 13-17 wt. % nickel, 6-8 wt. % molybdenum), and L605 alloy (18-22 wt. % chromium, 14-16 wt. % W, 9-11 wt. % nickel, balance cobalt). TiAlV alloys are also used in many medical devices (e.g., Ti-6Al-4V; 5.5-6.5 wt. % Al, 3.5-4.5 wt. % V and balance Ti; 3.5-4.5 wt. % vanadium, 5.5-6.75 wt. % aluminum, 0.3 wt. % max iron, 0.2 wt. % max oxygen, 0.08 wt. % max carbon, 0.05 wt. % max nitrogen, 0.015 wt. % max hydrogen H, 0.05 wt. % max yttrium, balance titanium).

Also, traditional materials such as stainless steel (316L) and cobalt-chromium alloys (e.g., MP35N, etc.) have a degree of recoil after being crimped and expanded that can interfere with obtaining a minimum crimping diameter and/or can adversely affect the placement of the expandable cardiovascular device at a treatment area. During a crimping process, a crimping device is typically used to crimp the cardiovascular device onto a catheter. After an initial crimping process, tradition materials such as stainless steel and cobalt-chromium alloys recoil to a larger diameter by 9+% of the minimum crimped diameter. As such, the cardiovascular device must be crimped multiple times onto a catheter to attempt to obtain a smaller crimped diameter on the catheter. However, subjecting the cardiovascular device to multiple crimpings can result in damage to the cardiovascular device (e.g., damage to the frame and/or struts of the cardiovascular device, damage to leaflets on an expandable heart valve, etc.). Likewise, when the cardiovascular device is expanded at a treatment area, the traditional materials of the cardiovascular device will recoil 9+% of the maximum expanded diameter. As such, the inflatable balloon on the catheter must be pressurized multiple times to repeatedly expand the cardiovascular device at the treatment area to ensure proper expansion of the cardiovascular device. However, subjecting the cardiovascular device to multiple balloon expansions can result in damage to the cardiovascular device (e.g., damage or breakage of a frame and/or strut, etc.) and/or damage to the treatment area (e.g., rupture of blood vessel, tear and/or puncture of tissue of an organ, etc.).

In view of the current state of the art of medical devices, there is a need for an improved medical device that a) produces less recoil compared to medical devices formed of stainless steel, cobalt-chromium alloys, or TiAlV alloys, and b) can form smaller crimping diameters compared to medical devices formed of stainless steel, cobalt-chromium alloys, or TiAlV alloys.

### SUMMARY OF THE DISCLOSURE

The present disclosure is direct to a medical device that is at least partially made of a metal alloy. The medical device can include an orthopedic device, PFO (patent foramen ovale) device, stent, valve (e.g., heart valve, TAVR valve, mitral valve replacement, tricuspid valve replacement, pulmonary valve replacement, etc.), spinal implant, frame and other structures for use with a spinal implant, vascular implant, graft, guide wire, sheath, catheter, needle, stent catheter, electrophysiology catheter, hypotube, staple, cutting device, any type of implant, pacemaker, dental implant, dental crown, dental braces, wire used in medical procedures, bone implant, artificial disk, artificial spinal disk, prosthetic implant or device to repair, replace and/or support a bone (e.g., acromion, atlas, axis, calcaneus, carpus, clavicle, coccyx, epicondyle, epitrochlea, femur, fibula, frontal bone, greater trochanter, humerus, ilium, ischium, mandible, maxilla, metacarpus, metatarsus, occipital bone, olecranon, parietal bone, patella, phalanx, radius, ribs, sacrum, scapula, sternum, talus, tarsus, temporal bone, tibia, ulna, zygomatic bone, etc.) and/or cartilage, bone plate nail, rod, screw, post, cage, plate, pedicle screw, cap, hinge, joint system, anchor, spacer, shaft, anchor, disk, ball, tension band, locking connector other structural assembly that is used in a body to support a structure, mount a structure, and/or repair a structure in a body such as, but not limited to, a human body, animal body, etc. In one non-limiting embodiment, the medical device includes an expandable frame (e.g., stent, prosthetic heart valve, etc.) that can plastically deform radially outwardly by an expansion arrangement (e.g., inflatable balloon, etc.). In another non-limiting embodiment, the metal alloy is not a self-expanding alloy.

In accordance with another and/or alternative non-limiting aspect of the present disclosure, there is provided a medical device that is partially or fully formed of metal alloy such as, but not limited to, modified titanium alloys, modified cobalt-chromium alloys, modified zirconium alloys, modified niobium alloys, refractory metal alloys, metal alloys that include at least 15 awt. % rhenium [e.g., standard stainless steel alloys that include at least 15 awt. % rhenium, standard CoCr alloys that include at least 15 awt. % rhenium, standard TiAlV alloys that include at least 15 awt. % rhenium, standard Al alloys that include at least 15 awt. % rhenium, standard Ni alloys that include at least 15 awt. % rhenium, standard Ti alloys that include at least 15 awt. % rhenium, standard W alloys that include at least 15 awt. % rhenium, standard Mo alloys that include at least 15 awt. % rhenium, standard Cu alloys that include at least 15 awt. % rhenium].

As defined herein, a standard stainless steel includes 10-28 wt. % chromium, 0-35 wt. % nickel, 0-4 wt. % molybdenum, 0-2 wt. % manganese, 0-0.75 wt. % silicon, 0-0.3 wt. % carbon, 0-5 wt. % titanium, 0-10 wt. % Cb, 0-5 wt. % copper, 0-4 wt. % aluminum, 0-10 wt. % tantalum, 0-1 wt. % Se, 0-2 wt. % V, 0-2 wt. % tungsten, 0-2 wt. % Nb, and at least 50 wt. % iron.

As defined herein, standard CoCr alloy includes 15-32 wt. % chromium, 1-36 wt. % nickel, 2-18 wt. % molybdenum, 0-18 wt. % iron, 0-1 wt. % titanium, 0-0.15 wt. % manganese, 0-0.15 wt. % silver, 0-0.025 wt. % carbon, 0-16 wt. % tungsten, 0-2 wt. % Si, 0-2 wt. % Al, 0-1 wt. % Fe, 30-68 wt % cobalt.

As defined herein, standard TiAlV alloy includes 5.5-6.75 wt. % Al, 3.5-4.5 wt. % V, 85-93 wt. % Ti, 0-0.4 wt. % iron, 0-0.2 wt. % carbon.

As defined herein standard Al alloy includes 80-99 wt. % Al, 0-12 wt. % Si, 0-5 wt. % Mg, 0-1 wt. % Mn, 0-0.5 wt. % Sc, 0-0.5 wt. % Be, 0-0.5 wt. % Y, 0-0.5 wt. % Ce, 0-0.5 wt. % Cr, 0-3 wt. % Fe, 0-0.5, 0-9 wt. % Zn, 0-0.5 wt. % Ti, 0-3 wt. % Li, 0-0.5 wt. % Ag, 0-0.5 wt. % Ca, 0-0.5 wt. % Zr, 0-1 wt. % Pb, 0-0.5 wt. % Cd, 0-0.05 wt. % Bi, 0-1 wt. % Ni, 0-0.2 wt. % V, 0-0.1 wt. % Ga, and 0-7 wt. % Cu.

As defined herein, standard Ni alloy includes 30-98 wt. % Ni, 5-25 wt. % Cr, 0-65 wt. % Fe, 0-30 wt. % Mo, 0-32 wt. % Cu, 0-32 wt. % Co, 2-2 wt. % Al, 0-6 wt. % Ta, 0-15 wt. % W, 0-5 wt. % Ti, 0-6 wt. % Nb, 0-3 wt. % Si.

As defined herein, standard Ti alloy includes 80-99 wt. % Ti, 0-6 wt. % Al, 0-3 wt. % Sn, 0-1 wt. % Pd, 0-8 wt. % V, 0-15 wt. % Mo, 0-1 wt. % Ni, 0-0.3 wt. % Ru, 0-6 wt. % Cr, 0-4 wt. % Zr, 0-4 wt. % Nb, 0-1 wt. % Si, 0.0.5 wt. % Co, 0-2 wt. % Fe.

As defined herein, standard W alloy includes 85-98 wt. % W, 0-8 wt. % Ni, 0-5 wt. % Cu, 0-5 wt. % Mo, 0-4 wt. % Fe.

As defined herein, standard Mo alloy includes 90-99.5 wt. % Mo, 0-1 wt. % Ni, 0-1 wt. % Ti, 0-1 wt. % Zr, 0-30 wt. % W, 0-2 wt. % Hf, 0-2 wt. % La.

As defined herein standard Cu alloy includes 55-95 wt. % Cu, 0-40 wt. % Zn, 0-10 wt. % Sn, 0-10 wt. % Pb, 0-1 wt. % Fe, 0-5 wt. % Si, 0-12 wt. % Mn, 0-12 wt. % Al, 0-3 wt. % Be, 0-1 Wt. % Co, 0-20 wt. % Ni.

As defined herein, a refractory metal alloy is a metal alloy that includes at least 20 wt. % of one or more of molybdenum, rhenium, niobium, tantalum or tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc.

Several non-limiting examples of metal alloys that can be used to partially or fully form the frame of the medical device are set forth below in weight percent:

| | Wt. % | | | |
|---|---|---|---|---|
| Component | Ex. 1 | Ex. 2 | Ex. 3 | Ex. 4 |
| Ag | 0-40% | 0-40% | 0-40% | 0-40% |
| Al | 0-40% | 0-40% | 0-40% | 0-40% |
| Bi | 0-40% | 0-40% | 0-40% | 0-40% |
| Cr | 0-40% | 0-40% | 0-40% | 0-40% |
| Cu | 0-40% | 0-40% | 0-40% | 0-40% |
| Co | 0-60% | 0-60% | 0-60% | 0-60% |
| Fe | 0-80% | 0-80% | 0-80% | 0-80% |
| Hf | 0-40% | 0-40% | 0-40% | 0-40% |
| Ir | 0-40% | 0-40% | 0-40% | 0-40% |
| Mg | 0-40% | 0-40% | 0-40% | 0-40% |
| Mn | 0-40% | 0-40% | 0-40% | 0-40% |
| Mo | 10-98% | 20-95% | 30-95% | 40-95% |
| Nb | 0-80% | 0-80% | 0-80% | 0-80% |
| Ni | 0-60% | 0-60% | 0-60% | 0-60% |
| Os | 0-40% | 0-40% | 0-40% | 0-40% |
| Pt | 0-40% | 0-40% | 0-40% | 0-40% |
| Re | 5-98% | 10-90% | 20-80% | 30-70% |
| Rh | 0-40% | 0-40% | 0-40% | 0-40% |
| Si | 0-40% | 0-40% | 0-40% | 0-40% |
| Sn | 0-40% | 0-40% | 0-40% | 0-40% |
| Ta | 0-80% | 0-60% | 0-80% | 0-80% |
| To | 0-40% | 0-40% | 0-40% | 0-40% |
| Ti | 0-60% | 0-60% | 0-60% | 0-60% |
| V | 0-40% | 0-40% | 0-40% | 0-40% |
| W | 0-98% | 0-98% | 0-98% | 0-98% |
| Y | 0-40% | 0-40% | 0-40% | 0-40% |
| Zr | 0-40% | 0-40% | 0-40% | 0-40% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06 | <0.06 | <0.06 | <0.06 |
| N | <0.06 | <0.06 | <0.06 | <0.06 |
| O | <0.06 | <0.06 | <0.06 | <0.06 |

| | Wt. % | | | |
|---|---|---|---|---|
| Component | Ex. 5 | Ex. 6 | Ex. 7 | Ex. 8 |
| Ag | 0-20% | 0-20% | 0-20% | 0-20% |
| Al | 0-35% | 0-30% | 5-30% | 0-25% |
| Bi | 0-20% | 0-20% | 0-20% | 0-20% |
| Cr | 10-40% | 0-40% | 0-40% | 0-40% |
| Cu | 0-20% | 0-20% | 0-20% | 0-20% |
| Co | 10-60% | 0-60% | 0-60% | 0-60% |
| Fe | 0-80% | 30-80% | 0-80% | 0-70% |
| Hf | 0-20% | 0-20% | 0-20% | 0-20% |
| Ir | 0-20% | 0-20% | 0-20% | 0-20% |
| Mg | 0-20% | 0-20% | 0-20% | 0-20% |
| Mn | 0-20% | 0-40% | 0-20% | 0-20% |
| Mo | 0-60% | 0-60% | 0-80% | 0-70% |
| Nb | 0-60% | 0-60% | 0-65% | 20-60% |
| Ni | 0-60% | 5-55% | 0-52% | 0-50% |
| Os | 0-20% | 0-20% | 0-20% | 0-20% |
| Pt | 0-20% | 0-20% | 0-20% | 0-20% |
| Re | 4.5-98% | 4.5-90% | 4.5-80% | 4.5-70% |
| Rh | 0-20% | 0-20% | 0-20% | 0-20% |
| Si | 0-20% | 0-20% | 0-20% | 0-20% |
| Sn | 0-20% | 0-20% | 0-20% | 0-20% |
| Ta | 0-60% | 0-60% | 5-65% | 0-60% |
| Tc | 0-20% | 0-20% | 0-20% | 0-20% |
| Ti | 0-60% | 0-55% | 0-53% | 0-50% |
| V | 0-20% | 0-20% | 2-20% | 0-20% |
| W | 0-60% | 0-60% | 0-80% | 0-70% |
| Y | 0-20% | 0-20% | 0-20% | 0-20% |
| Zr | 0-20% | 0-20% | 0-20% | 5-20% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06 | <0.06 | <0.06 | <0.06 |
| N | <0.06 | <0.06 | <0.06 | <0.06 |
| O | <0.06 | <0.06 | <0.06 | <0.06 |

| | Wt. % | | | |
|---|---|---|---|---|
| Component | Ex. 9 | Ex. 10 | Ex. 11 | Ex. 12 |
| Ag | 0-5% | 0-5% | 0-5% | 0-5% |
| Al | 0-5% | 0-5% | 1-15% | 0-20% |
| Bi | 0-5% | 0-5% | 0-5% | 0-5% |
| Cr | 1-28% | 1-30% | 0-5% | 0-30% |
| Cu | 0-20% | 0-5% | 0-5% | 0-25% |
| Co | 0-5% | 1-60% | 0-5% | 0-60% |
| Fe | 10-80% | 0-25% | 0-5% | 0-80% |
| Hf | 0-5% | 0-5% | 0-5% | 0-5% |
| Ir | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-8% | 0-25% | 0-5% | 0-98% |
| Nb | 0-5% | 0-5% | 0-5% | 0-95% |
| Ni | 1-20% | 1-45% | 0-5% | 0-50% |
| Os | 0-5% | 0-5% | 0-5% | 0-5% |
| Pt | 0-5% | 0-5% | 0-5% | 0-5% |
| Re | 5-20% | 4.8-20% | 4.5-20% | 4.5-20% |
| Rh | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 0-98% |
| Tc | 0-5% | 0-5% | 0-5% | 0-5% |
| Ti | 0-5% | 0-5% | 40-93% | 0-93% |
| V | 0-5% | 0-5% | 1-10% | 0-20% |
| W | 0-5% | 0-20% | 0-5% | 0-98% |
| Y | 0-5% | 0-5% | 0-5% | 0-5% |
| Zr | 0-5% | 0-5% | 0-5% | 0-5% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06 | <0.06 | <0.06 | <0.06 |
| N | <0.06 | <0.06 | <0.06 | <0.06 |
| O | <0.06 | <0.06 | <0.06 | <0.06 |

| | Wt. % | | | |
|---|---|---|---|---|
| Component | Ex. 13 | Ex. 14 | Ex. 15 | Ex. 16 |
| Mo | 40-80% | 40-80% | 40-80% | 40-80% |
| C | 0.01-0.3% | 0-0.3% | 0-0.3% | 0-0.3% |
| Co | ≤0.002% | ≤0.002% | ≤0.002% | ≤0.002% |
| Cs₂O | 0-0.2% | 0-0.2% | 0.01-0.2% | 0-0.2% |
| Fe | ≤0.02% | ≤0.02% | ≤0.02% | ≤0.02% |
| H | ≤0.002% | ≤0.002% | ≤0.002% | ≤0.002% |
| Hf | 0.1-2.5% | 0-2.5% | 0-2.5% | 0-2.5% |
| O | ≤0.06% | ≤0.06% | ≤0.06% | ≤0.06% |
| Os | ≤1% | ≤1% | ≤1% | ≤1% |
| La₂O₃ | 0-32% | 0.1-2% | 0-2% | 0-2% |
| N | ≤20 ppm | ≤20 ppm | ≤20 ppm | ≤20 ppm |
| Nb | ≤0.01% | ≤0.01% | ≤0.01% | ≤0.01% |
| Pt | ≤1% | ≤1% | ≤1% | ≤1% |
| Re | 7-49% | 7.5-49% | 7.5-49% | 7.5-49% |
| S | ≤0.008% | ≤0.008% | ≤0.008% | ≤0.008% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| W | 0-50% | 0-50% | 0-50% | 0.5-50% |
| Y₂O₃ | 0-1% | 0-1% | 0.1-1% | 0-1% |
| Zr | ≤1% | ≤1% | ≤1% | ≤1% |
| ZrO₂ | 0-3% | 0-3% | 0-3% | 0-3% |
| CNT | 0-10% | 0-10% | 0-10% | 0-10% |
| C | <0.06 | <0.06 | <0.06 | <0.06 |
| N | <0.06 | <0.06 | <0.06 | <0.06 |
| O | <0.06 | <0.06 | <0.06 | <0.06 |

| | Wt. % | | |
|---|---|---|---|
| Component | Ex. 17 | Ex. 18 | Ex. 19 |
| Mo | 40-80% | 40-80% | 40-80% |
| C | 0-0.3% | 0-0.3% | 0-0.3% |
| Co | ≤0.002% | ≤0.002% | ≤0.002% |
| Cs₂O | 0-0.2% | 0-0.2% | 0-0.2% |
| H | ≤0.002% | ≤0.002% | ≤0.002% |
| Hf | 0-2.5% | 0-2.5% | 0-2.5% |
| O | ≤0.06% | ≤0.06% | ≤0.06% |
| Os | ≤1% | ≤1% | ≤1% |
| La₂O₃ | 0-2% | 0-2% | 0-2% |
| N | ≤20 ppm | ≤20 ppm | ≤20 ppm |
| Nb | ≤0.01% | ≤0.01% | ≤0.01% |
| Pt | ≤1% | ≤1% | ≤1% |
| Re | 7-49% | 7.5-49% | 7.5-49% |
| S | ≤0.008% | ≤0.008% | ≤0.008% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| W | 0-50% | 0-50% | 0-50% |
| Y₂O₃ | 0-1% | 0-1% | 0-1% |
| ZrO₂ | 0.1-3% | 0-3% | 0-3% |
| CNT | 0-10% | 0-10% | 0-10% |
| C | <0.06 | <0.06 | <0.06 |
| N | <0.06 | <0.06 | <0.06 |
| O | <0.06 | <0.06 | <0.06 |

| | Wt. % | | |
|---|---|---|---|
| Component | Ex. 20 | Ex. 21 | Ex. 22 |
| Mo | 45-78% | 45-75% | 45-70% |
| C | 0-0.3% | 0-0.3% | 0-0.3% |
| Co | ≤0.002% | ≤0.002% | ≤0.002% |
| Cs₂O | 0-0.2% | 0-0.2% | 0-0.2% |
| H | ≤0.002% | ≤0.002% | ≤0.002% |
| Hf | 0-2.5% | 0-2.5% | 0-2.5% |
| O | ≤0.06% | ≤0.06% | ≤0.06% |
| Os | ≤1% | ≤1% | ≤1% |
| La₂O₃ | 0-2% | 0-2% | 0-2% |
| N | ≤20 ppm | ≤20 ppm | ≤20 ppm |
| Nb | ≤0.01% | ≤0.01% | ≤0.01% |
| Pt | ≤1% | ≤1% | ≤1% |
| Re | 7-49% | 7.5-49% | 7.5-49% |
| S | ≤0.008% | ≤0.008% | ≤0.008% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| W | 0-50% | 0-50% | 0-50% |
| Y₂O₃ | 0-1% | 0-1% | 0-1% |
| ZrO₂ | 0.1-3% | 0-3% | 0-3% |
| CNT | 0-10% | 0-10% | 0-10% |
| C | <0.06 | <0.06 | <0.06 |
| N | <0.06 | <0.06 | <0.06 |
| O | <0.06 | <0.06 | <0.06 |

| | Wt. % | | | |
|---|---|---|---|---|
| Component | Ex. 23 | Ex. 24 | Ex. 25 | Ex. 26 |
| Mo | 35-80% | 35-80% | 35-70% | 35-65% |
| C | 0.05-0.15% | 0-0.15% | 0-0.15% | 0-0.15% |
| Cs₂O | 0-0.2% | 0-0.2% | 0.04-0.1% | 0-0.2% |
| Hf | 0.8-1.4% | 0-2% | 0-2.5% | 0-2.5% |
| La₂O₃ | 0-2% | 0.3-0.7% | 0-2% | 0-2% |
| Re | 7-49% | 7-49% | 7.5-49% | 7.5-49% |
| Ta | 0-2% | 0-2% | 0-50% | 0-50% |
| W | 0-2% | 0-2% | 0-50% | 20-50% |
| Y₂O₃ | 0-1% | 0-1% | 0.3-0.5% | 0-1% |
| ZrO₂ | 0-3% | 0-3% | 0-3% | 0-3% |

Wt. %

| Component | Ex. 27 | Ex. 28 | Ex. 29 |
|---|---|---|---|
| Mo | 40-60% | 35-60% | 30-60% |
| C | 0-0.15% | 0-0.15% | 0-0.15% |
| Cs₂O | 0-0.2% | 0-0.2% | 0-0.2% |
| Hf | 0-2.5% | 0-2.5% | 0-2.5% |
| La₂O₃ | 0-2% | 0-2% | 0-2% |
| Re | 7-60% | 7.5-65% | 7.5-70% |
| Ta | 0-3% | 10-50% | 0-40% |
| W | 0-3% | 0-50% | 0-40% |
| Y₂O₃ | 0-1% | 0-1% | 0-1% |
| ZrO₂ | 1.2-1.8% | 0-3% | 0-3% |

Wt. %

| Component | Ex. 30 | Ex. 31 | Ex. 32 |
|---|---|---|---|
| W | 20-80% | 60-80% | 20-78% |
| Re | 7.5-47.5% | 10-40% | 8-47.5% |
| Mo | 0-47.5% | <0.5% | 1-47.5% |
| Cu | <0.5% | <0.5% | <0.5% |
| C | ≤0.15% | ≤0.15% | ≤0.15% |
| Co | ≤0.002% | ≤0.002% | ≤0.002% |
| Cs₂O | ≤0.2% | ≤0.2% | ≤0.2% |
| Fe | ≤0.02% | ≤0.02% | ≤0.02% |
| H | ≤0.002% | ≤0.002% | ≤0.002% |
| Hf | <0.5% | <0.5% | <0.5% |
| La₂O₃ | <0.5% | <0.5% | <0.5% |
| O | ≤0.06% | ≤0.06% | ≤0.06% |
| Os | <0.5% | <0.5% | <0.5% |
| N | ≤20 ppm | ≤20 ppm | ≤20 ppm |
| Nb | ≤0.01% | ≤0.01% | ≤0.01% |
| Pt | <0.5% | <0.5% | <0.5% |
| S | ≤0.008% | ≤0.008% | ≤0.008% |
| Sn | ≤0.002% | ≤0.002% | ≤0.002% |
| Ta | <0.5% | <0.5% | <0.5% |
| Tc | <0.5% | <0.5% | <0.5% |
| Ti | <0.5% | <0.5% | <0.5% |
| V | <0.5% | <0.5% | <0.5% |
| Y₂O₃ | <0.5% | <0.5% | <0.5% |
| Zr | <0.5% | <0.5% | <0.5% |
| ZrO₂ | <0.5% | <0.5% | <0.5% |
| CNT | 0-10% | 0-10% | <0.5%. |
| C | <0.06 | <0.06 | <0.06 |
| N | <0.06 | <0.06 | <0.06 |
| O | <0.06 | <0.06 | <0.06 |

Wt. %

| Component | Ex. 33 | Ex. 34 | Ex. 35 |
|---|---|---|---|
| W | 20-80% | 60-80% | 20-75% |
| Re | 7.5-47.5% | 10-40% | 7.5-47.5% |
| Mo | 0-47.5% | <0.5% | 1-47.5% |

| | Wt. % | | |
|---|---|---|---|
| Component | Ex. 36 | Ex. 37 | Ex. 38 |
| W | 50.1-80% | 65-80% | 50.1-79% |
| Re | 10-40% | 10-35% | 10-40% |
| Mo | 0-40% | <0.5% | 1-30% |

| | Wt. % | | |
|---|---|---|---|
| Component | Ex. 39 | Ex. 40 | Ex. 41 |
| W | 20-49% | 20-49% | 20-49% |
| Re | 7.5-60% | 7.5-60% | 7.5-60% |
| Mo | 0-40% | 0-40% | 0-39% |

| | Wt. % | | |
|---|---|---|---|
| Component | Ex. 42 | Ex. 43 | Ex. 44 |
| Re | 5-98% | 60-95% | 80-90% |
| Mo | 0-80% | 0-40% | 0-20% |
| W | 0-80% | 0-40% | 0-20% |

| | Wt. % | | |
|---|---|---|---|
| Component | Ex. 45 | Ex. 46 | Ex. 47 |
| W | 20-49% | 20-49% | 20-49% |
| Re | 6-40% | 6-40% | 6-39% |
| Mo | 20-60% | 30-60% | 40-60% |

| | Wt. % | | |
|---|---|---|---|
| Component | Ex. 48 | Ex. 49 | Ex. 50 |
| W | 20-40% | 20-35% | 20-30% |
| Re | 6-40% | 6-40% | 6-40% |
| Mo | 0-40% | 10-40% | 31-40% |

Wt. %

| Component | Ex. 51 | Ex. 52 | Ex. 53 | Ex. 54 |
|---|---|---|---|---|
| Re | 5-60% | 5-60% | 5-60% | 5-60% |
| Mo | 0-55% | 10-55% | 10-55% | 10-55% |
| Bi | 1-42 | 0-32 | 0-32 | 0-32 |
| Cr | 0-32 | 1-42 | 0-32 | 0-32 |
| Ir | 0-32 | 0-32 | 1-42 | 0-32 |
| Nb | 0-32 | 0-32 | 0-32 | 1-42 |
| Ta | 0-32 | 0-32 | 0-32 | 0-32 |
| Ti | 0-32 | 0-32 | 0-32 | 0-32 |
| Y | 0-32 | 0-32 | 0-32 | 0-32 |
| Zr | 0-32 | 0-32 | 0-32 | 0-32 |
| C | <0.06 | <0.06 | <0.06 | <0.06 |
| N | <0.06 | <0.06 | <0.06 | <0.06 |
| O | <0.06 | <0.06 | <0.06 | <0.06 |

Wt. %

| Component | Ex. 55 | Ex. 56 | Ex. 57 | Ex. 58 |
|---|---|---|---|---|
| Re | 5-60% | 5-60% | 5-60% | 5-60% |
| Mo | 15-55% | 15-55% | 15-55% | 15-55% |
| Bi | 0-32 | 0-32 | 0-32 | 0-32 |
| Cr | 0-32 | 0-32 | 0-32 | 0-32 |
| Ir | 0-32 | 0-32 | 0-32 | 0-32 |
| Nb | 0-32 | 0-32 | 0-32 | 0-32 |
| Ta | 1-42 | 0-32 | 0-32 | 0-32 |
| Ti | 0-32 | 1-42 | 0-32 | 0-32 |
| Y | 0-32 | 0-32 | 1-42 | 0-32 |
| Zr | 0-32 | 0-32 | 0-32 | 1-42 |
| C | <0.06 | <0.06 | <0.06 | <0.06 |
| N | <0.06 | <0.06 | <0.06 | <0.06 |
| O | <0.06 | <0.06 | <0.06 | <0.06 |

Wt. %

| Component | Ex. 59 | Ex. 60 | Ex. 61 | Ex. 62 |
|---|---|---|---|---|
| Re | 41-59% | 41-59% | 41-59% | 41-59% |
| Mo | 18-45% | 18-45% | 18-45% | 18-45% |
| Bi | 1-42 | 0-32 | 0-32 | 0-32 |
| Cr | 0-32 | 1-42 | 0-32 | 0-32 |
| Ir | 0-32 | 0-32 | 1-42 | 0-32 |
| Nb | 0-32 | 0-32 | 0-32 | 1-42 |
| Ta | 0-32 | 0-32 | 0-32 | 0-32 |
| Ti | 0-32 | 0-32 | 0-32 | 0-32 |
| Y | 0-32 | 0-32 | 0-32 | 0-32 |
| Zr | 0-32 | 0-32 | 0-32 | 0-32 |
| C | <0.06 | <0.06 | <0.06 | <0.06 |
| N | <0.06 | <0.06 | <0.06 | <0.06 |
| O | <0.06 | <0.06 | <0.06 | <0.06 |

Wt. %

| Component | Ex. 63 | Ex. 64 | Ex. 65 | Ex. 66 |
|---|---|---|---|---|
| Re | 41-59% | 41-59% | 41-59% | 41-59% |
| Mo | 18-45% | 18-45% | 18-45% | 18-45% |
| Bi | 0-32 | 0-32 | 0-32 | 0-32 |
| Cr | 0-32 | 0-32 | 0-32 | 0-32 |
| Ir | 0-32 | 0-32 | 0-32 | 0-32 |
| Nb | 0-32 | 0-32 | 0-32 | 0-32 |
| Ta | 1-42 | 0-32 | 0-32 | 0-32 |
| Ti | 0-32 | 1-42 | 0-32 | 0-32 |
| Y | 0-32 | 0-32 | 1-42 | 0-32 |
| Zr | 0-32 | 0-32 | 0-32 | 1-42 |
| C | <0.06 | <0.06 | <0.06 | <0.06 |
| N | <0.06 | <0.06 | <0.06 | <0.06 |
| O | <0.06 | <0.06 | <0.06 | <0.06 |

Wt. %

| Component | Ex. 67 | Ex. 68 | Ex. 69 | Ex. 70 |
|---|---|---|---|---|
| Re | 41-59% | 41-59% | 41-59% | 41-59% |
| Mo | 18-45% | 18-45% | 18-45% | 18-45% |
| Bi | 0-15 | 0-15 | 1-36 | 0-15 |
| Cr | 1-20 | 1-20 | 1-20 | 1-20 |
| Ir | 0-15 | 0-15 | 0-15 | 0-15 |
| Nb | 1-36 | 0-15 | 0-15 | 0-15 |
| Ta | 0-15 | 1-36 | 0-15 | 0-15 |
| Ti | 0-15 | 0-15 | 0-15 | 0-15 |
| Y | 0-15 | 0-15 | 0-15 | 0-15 |
| Zr | 0-15 | 0-15 | 0-15 | 1-36 |
| C | <0.06 | <0.06 | <0.06 | <0.06 |
| N | <0.06 | <0.06 | <0.06 | <0.06 |
| O | <0.06 | <0.06 | <0.06 | <0.06 |

Wt. %

| Component | Ex. 71 | Ex. 72 | Ex. 73 | Ex. 74 |
|---|---|---|---|---|
| Re | 41-59% | 41-59% | 41-59% | 41-59% |
| Mo | 18-45% | 18-45% | 18-45% | 18-45% |
| Bi | 1-36 | 0-15 | 0-15 | 0-15 |
| Cr | 1-20 | 1-20 | 1-20 | 1-20 |
| Ir | 0-15 | 1-36 | 0-15 | 0-15 |
| Nb | 0-15 | 0-15 | 0-15 | 0-15 |
| Ta | 0-15 | 0-15 | 0-15 | 0-15 |
| Ti | 0-15 | 0-15 | 1-36 | 0-15 |
| Y | 0-15 | 0-15 | 0-15 | 1-36 |
| Zr | 0-15 | 0-15 | 0-15 | 0-15 |
| C | <0.06 | <0.06 | <0.06 | <0.06 |
| N | <0.06 | <0.06 | <0.06 | <0.06 |
| O | <0.06 | <0.06 | <0.06 | <0.06 |

Wt. %

| Component | Ex. 75 | Ex. 76 | Ex. 77 | Ex. 78 |
|---|---|---|---|---|
| Re | 41-59% | 41-59% | 41-59% | 41-59% |
| Mo | 18-45% | 18-45% | 18-45% | 18-45% |
| Bi | 1-34 | 0-15 | 0-15 | 0-15 |
| Cr | 0-15 | 0-15 | 0-15 | 0-15 |
| Ir | 0-15 | 0-15 | 0-15 | 1-34 |
| Nb | 3-27 | 3-27 | 3-27 | 3-27 |
| Ta | 0-42 | 1-34 | 0-15 | 0-15 |
| Ti | 0-15 | 0-15 | 0-15 | 0-15 |
| Y | 0-15 | 0-15 | 0-15 | 0-15 |
| Zr | 0-15 | 0-15 | 3-27 | 0-15 |
| C | <0.06 | <0.06 | <0.06 | <0.06 |
| N | <0.06 | <0.06 | <0.06 | <0.06 |
| O | <0.06 | <0.06 | <0.06 | <0.06 |

Wt. %

| Component | Ex. 79 | Ex. 80 | Ex. 81 | Ex. 82 |
|---|---|---|---|---|
| Re | 41-59% | 41-59% | 41-59% | 41-59% |
| Mo | 18-45% | 18-45% | 18-45% | 18-45% |
| Bi | 0-15 | 0-15 | 0-15 | 0-15 |
| Cr | 0-15 | 0-15 | 0-15 | 0-15 |
| Ir | 0-15 | 1-34 | 0-15 | 0-15 |
| Nb | 0-15 | 0-15 | 0-15 | 0-15 |
| Ta | 1-34 | 0-15 | 3-27 | 0-15 |
| Ti | 0-15 | 0-15 | 0-15 | 0-15 |
| Y | 0-15 | 0-15 | 0-15 | 3-27 |
| Zr | 3-27 | 3-27 | 3-27 | 3-27 |
| C | <0.06 | <0.06 | <0.06 | <0.06 |
| N | <0.06 | <0.06 | <0.06 | <0.06 |
| O | <0.06 | <0.06 | <0.06 | <0.06 |

Wt. %

| Component | Ex. 83 | Ex. 84 | Ex. 85 | Ex. 86 |
|---|---|---|---|---|
| Re | 41-59% | 41-59% | 41-59% | 41-59% |
| Mo | 18-45% | 18-45% | 18-45% | 18-45% |
| Bi | 0-15 | 0-15 | 0-15 | 0-15 |
| Cr | 0-15 | 0-15 | 0-15 | 1-10 |
| Ir | 1-34 | 0-25 | 3-27 | 0-15 |
| Nb | 0-15 | 3-27 | 0-15 | 0-15 |
| Ta | 0-15 | 0-15 | 1-34 | 0-15 |
| Ti | 0-15 | 0-15 | 0-15 | 0-15 |
| Y | 3-27 | 3-27 | 0-15 | 0-15 |
| Zr | 0-15 | 0-15 | 3-27 | 1-12 |
| C | <0.06 | <0.06 | <0.06 | <0.06 |
| N | <0.06 | <0.06 | <0.06 | <0.06 |
| O | <0.06 | <0.06 | <0.06 | <0.06 |

| | Wt. % | | | |
|---|---|---|---|---|
| Component | Ex. 87 | Ex. 88 | Ex. 89 | Ex. 90 |
| Re | 50-75% | 55-75% | 60-75% | 65-75% |
| Cr | 25-50% | 25-45% | 25-40% | 25-35% |
| Mo | 0-25% | 0-25% | 0-25% | 0-25% |
| Bi | 0-25% | 0-25% | 0-25% | 0-25% |
| Cr | 0-25% | 0-25% | 0-25% | 0-25% |
| Ir | 0-25% | 0-25% | 0-25% | 0-25% |
| Nb | 0-25% | 0-25% | 0-25% | 0-25% |
| Ta | 0-25% | 0-25% | 0-25% | 0-25% |
| V | 0-25% | 0-25% | 0-25% | 0-25% |
| W | 0-25% | 0-25% | 0-25% | 0-25% |
| Mn | 0-25% | 0-25% | 0-25% | 0-25% |
| Tc | 0-25% | 0-25% | 0-25% | 0-25% |
| Ru | 0-25% | 0-25% | 0-25% | 0-25% |
| Rh | 0-25% | 0-25% | 0-25% | 0-25% |
| Hf | 0-25% | 0-25% | 0-25% | 0-25% |
| Os | 0-25% | 0-25% | 0-25% | 0-25% |
| Cu | 0-25% | 0-25% | 0-25% | 0-25% |
| Ir | 0-25% | 0-25% | 0-25% | 0-25% |
| Ti | 0-25% | 0-25% | 0-25% | 0-25% |
| Y | 0-25% | 0-25% | 0-25% | 0-25% |
| Zr | 0-25% | 0-25% | 0-25% | 0-25% |
| Ag | 0-25% | 0-25% | 0-25% | 0-25% |
| Al | 0-25% | 0-25% | 0-25% | 0-22% |
| Co | 0-25% | 0-25% | 0-25% | 0-25% |
| Fe | 0-25% | 0-25% | 0-25% | 0-25% |
| Mg | 0-25% | 0-25% | 0-25% | 0-25% |
| Ni | 0-25% | 0-25% | 0-25% | 0-25% |
| Pt | 0-25% | 0-25% | 0-25% | 0-25% |
| Si | 0-25% | 0-25% | 0-25% | 0-25% |
| Sn | 0-25% | 0-25% | 0-25% | 0-25% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06 | <0.06 | <0.06 | <0.06 |
| N | <0.06 | <0.06 | <0.06 | <0.06 |
| O | <0.06 | <0.06 | <0.06 | <0.06 |

| | Wt. % | | | |
|---|---|---|---|---|
| Component | Ex. 91 | Ex. 92 | Ex. 93 | Ex. 94 |
| Re | 50-72% | 55-72% | 60-72% | 65-72% |
| Cr | 28-50% | 28-45% | 28-40% | 28-35% |
| Mo | 0-25% | 0-25% | 0-25% | 0-25% |
| Bi | 0-10% | 0-10% | 0-10% | 0-10% |
| Cr | 0-10% | 0-10% | 0-10% | 0-10% |
| Ir | 0-10% | 0-10% | 0-10% | 0-10% |
| Nb | 0-10% | 0-10% | 0-10% | 0-10% |
| Ta | 0-10% | 0-10% | 0-10% | 0-10% |
| V | 0-10% | 0-10% | 0-10% | 0-10% |
| W | 0-10% | 0-10% | 0-10% | 0-10% |
| Mn | 0-10% | 0-10% | 0-10% | 0-10% |
| Tc | 0-10% | 0-10% | 0-10% | 0-10% |
| Ru | 0-10% | 0-10% | 0-10% | 0-10% |
| Rh | 0-10% | 0-10% | 0-10% | 0-10% |
| Hf | 0-10% | 0-10% | 0-10% | 0-10% |
| Os | 0-10% | 0-10% | 0-10% | 0-10% |
| Cu | 0-10% | 0-10% | 0-10% | 0-10% |
| Ir | 0-10% | 0-10% | 0-10% | 0-10% |
| Ti | 0-10% | 0-10% | 0-10% | 0-10% |
| Y | 0-10% | 0-10% | 0-10% | 0-10% |
| Zr | 0-10% | 0-10% | 0-10% | 0-10% |
| Ag | 0-10% | 0-10% | 0-10% | 0-10% |
| Al | 0-10% | 0-10% | 0-10% | 0-10% |
| Co | 0-10% | 0-10% | 0-10% | 0-10% |
| Fe | 0-10% | 0-10% | 0-10% | 0-10% |
| Mg | 0-10% | 0-10% | 0-10% | 0-10% |
| Ni | 0-10% | 0-10% | 0-10% | 0-10% |
| Pt | 0-10% | 0-10% | 0-10% | 0-10% |
| Si | 0-10% | 0-10% | 0-10% | 0-10% |
| Sn | 0-10% | 0-10% | 0-10% | 0-10% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06 | <0.06 | <0.06 | <0.06 |
| N | <0.06 | <0.06 | <0.06 | <0.06 |
| O | <0.06 | <0.06 | <0.06 | <0.06 |

| | Wt. % | | | |
|---|---|---|---|---|
| Component | Ex. 95 | Ex. 96 | Ex. 97 | Ex. 98 |
| Re | 50-70% | 55-70% | 60-70% | 65-70% |
| Cr | 30-50% | 30-45% | 30-40% | 30-35% |
| Mo | 0-10% | 0-10% | 0-10% | 0-10% |
| Bi | 0-10% | 0-10% | 0-10% | 0-10% |
| Cr | 0-10% | 0-10% | 0-10% | 0-10% |
| Ir | 0-10% | 0-10% | 0-10% | 0-10% |
| Nb | 0-10% | 0-10% | 0-10% | 0-10% |
| Ta | 0-10% | 0-10% | 0-10% | 0-10% |
| V | 0-10% | 0-10% | 0-10% | 0-10% |
| W | 0-10% | 0-10% | 0-10% | 0-10% |
| Mn | 0-10% | 0-10% | 0-10% | 0-10% |
| Tc | 0-10% | 0-10% | 0-10% | 0-10% |
| Ru | 0-10% | 0-10% | 0-10% | 0-10% |
| Rh | 0-10% | 0-10% | 0-10% | 0-10% |
| Hf | 0-10% | 0-10% | 0-10% | 0-10% |
| Os | 0-10% | 0-10% | 0-10% | 0-10% |
| Cu | 0-10% | 0-10% | 0-10% | 0-10% |
| Ir | 0-10% | 0-10% | 0-10% | 0-10% |
| Ti | 0-10% | 0-10% | 0-10% | 0-10% |
| Y | 0-10% | 0-10% | 0-10% | 0-10% |
| Zr | 0-10% | 0-10% | 0-10% | 0-10% |
| Ag | 0-10% | 0-10% | 0-10% | 0-10% |
| Al | 0-10% | 0-10% | 0-10% | 0-10% |
| Co | 0-10% | 0-10% | 0-10% | 0-10% |
| Fe | 0-10% | 0-10% | 0-10% | 0-10% |
| Mg | 0-10% | 0-10% | 0-10% | 0-10% |
| N | 0-10% | 0-10% | 0-10% | 0-10% |
| Pt | 0-10% | 0-10% | 0-10% | 0-10% |
| Si | 0-10% | 0-10% | 0-10% | 0-10% |
| Sn | 0-10% | 0-10% | 0-10% | 0-10% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06 | <0.06 | <0.06 | <0.06 |
| N | <0.06 | <0.06 | <0.06 | <0.06 |
| O | <0.06 | <0.06 | <0.06 | <0.06 |

| | Wt. % | | | |
|---|---|---|---|---|
| Component | Ex. 99 | Ex. 100 | Ex. 101 | Ex. 102 |
| Re | 50-67.5% | 55-67.5% | 60-67.5% | 65-67.5% |
| Cr | 32.5-50% | 32.5-45% | 32.5-40% | 32.5-35% |
| Mo | 0-10% | 0-10% | 0-10% | 0-10% |
| Bi | 0-10% | 0-10% | 0-10% | 0-10% |
| Cr | 0-10% | 0-10% | 0-10% | 0-10% |
| Ir | 0-10% | 0-10% | 0-10% | 0-10% |
| Nb | 0-10% | 0-10% | 0-10% | 0-10% |
| Ta | 0-10% | 0-10% | 0-10% | 0-10% |
| V | 0-10% | 0-10% | 0-10% | 0-10% |
| W | 0-10% | 0-10% | 0-10% | 0-10% |
| Mn | 0-10% | 0-10% | 0-10% | 0-10% |
| Tc | 0-10% | 0-10% | 0-10% | 0-10% |
| Ru | 0-10% | 0-10% | 0-10% | 0-10% |
| Rh | 0-10% | 0-10% | 0-10% | 0-10% |
| Hf | 0-10% | 0-10% | 0-10% | 0-10% |
| Os | 0-10% | 0-10% | 0-10% | 0-10% |
| Cu | 0-10% | 0-10% | 0-10% | 0-10% |
| Ir | 0-10% | 0-10% | 0-10% | 0-10% |
| Ti | 0-10% | 0-10% | 0-10% | 0-10% |
| Y | 0-10% | 0-10% | 0-10% | 0-10% |
| Zr | 0-10% | 0-10% | 0-10% | 0-10% |
| Ag | 0-10% | 0-10% | 0-10% | 0-10% |
| Al | 0-10% | 0-10% | 0-10% | 0-10% |
| Co | 0-10% | 0-10% | 0-10% | 0-10% |
| Fe | 0-10% | 0-10% | 0-10% | 0-10% |
| Mg | 0-10% | 0-10% | 0-10% | 0-10% |
| Ni | 0-10% | 0-10% | 0-10% | 0-10% |
| Pt | 0-10% | 0-10% | 0-10% | 0-10% |
| Si | 0-10% | 0-10% | 0-10% | 0-10% |
| Sn | 0-10% | 0-10% | 0-10% | 0-10% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06 | <0.06 | <0.06 | <0.06 |
| N | <0.06 | <0.06 | <0.06 | <0.06 |
| O | <0.06 | <0.06 | <0.06 | <0.06 |

| | Wt. % | | | |
|---|---|---|---|---|
| Component | Ex. 103 | Ex. 104 | Ex. 105 | Ex. 106 |
| Re | 50-67.5% | 55-67.5% | 60-67.5% | 65-67.5% |
| Cr | 32.5-50% | 32.5-45% | 32.5-40% | 32.5-35% |
| Mo | 0-5% | 0-5% | 0-5% | 0-5% |
| Bi | 0-5% | 0-5% | 0-5% | 0-5% |
| Cr | 0-5% | 0-5% | 0-5% | 0-5% |
| Ir | 0-5% | 0-5% | 0-5% | 0-5% |
| Nb | 0-5% | 0-5% | 0-5% | 0-5% |
| Ta | 0-5% | 0-5% | 0-5% | 0-5% |
| V | 0-5% | 0-5% | 0-5% | 0-5% |
| W | 0-5% | 0-5% | 0-5% | 0-5% |
| Mn | 0-5% | 0-5% | 0-5% | 0-5% |
| Tc | 0-5% | 0-5% | 0-5% | 0-5% |
| Ru | 0-5% | 0-5% | 0-5% | 0-5% |
| Rh | 0-5% | 0-5% | 0-5% | 0-5% |
| Hf | 0-5% | 0-5% | 0-5% | 0-5% |
| Os | 0-5% | 0-5% | 0-5% | 0-5% |
| Cu | 0-5% | 0-5% | 0-5% | 0-5% |
| Ir | 0-5% | 0-5% | 0-5% | 0-5% |
| Ti | 0-5% | 0-5% | 0-5% | 0-5% |
| Y | 0-5% | 0-5% | 0-5% | 0-5% |
| Zr | 0-5% | 0-5% | 0-5% | 0-5% |
| Ag | 0-5% | 0-5% | 0-5% | 0-5% |
| Al | 0-5% | 0-5% | 0-5% | 0-5% |
| Co | 0-5% | 0-5% | 0-5% | 0-5% |
| Fe | 0-5% | 0-5% | 0-5% | 0-5% |
| Mg | 0-5% | 0-5% | 0-5% | 0-5% |
| Ni | 0-5% | 0-5% | 0-5% | 0-5% |
| Pt | 0-5% | 0-5% | 0-5% | 0-5% |
| Si | 0-5% | 0-5% | 0-5% | 0-5% |
| Sn | 0-5% | 0-5% | 0-5% | 0-5% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06 | <0.06 | <0.06 | <0.06 |
| N | <0.06 | <0.06 | <0.06 | <0.06 |
| O | <0.06 | <0.06 | <0.06 | <0.06 |

| | Wt. % | | | |
|---|---|---|---|---|
| Component | Ex. 107 | Ex. 108 | Ex. 109 | Ex. 110 |
| Re | 50-75% | 55-72% | 60-70% | 62-70% |
| Cr | 24-49% | 27-44% | 29-39% | 29-37% |
| Mo | 1-15% | 1-10% | 1-8% | 1-5% |
| Bi | 0-15% | 0-10% | 0-8% | 0-5% |
| Cr | 0-15% | 0-10% | 0-8% | 0-5% |
| Ir | 0-15% | 0-10% | 0-8% | 0-5% |
| Nb | 0-15% | 0-10% | 0-8% | 0-5% |
| Ta | 0-15% | 0-10% | 0-8% | 0-5% |
| V | 0-15% | 0-10% | 0-8% | 0-5% |
| W | 0-15% | 0-10% | 0-8% | 0-5% |
| Mn | 0-15% | 0-10% | 0-8% | 0-5% |
| Tc | 0-15% | 0-10% | 0-8% | 0-5% |
| Ru | 0-15% | 0-10% | 0-8% | 0-5% |
| Rh | 0-15% | 0-10% | 0-8% | 0-5% |
| Hf | 0-15% | 0-10% | 0-8% | 0-5% |
| Os | 0-15% | 0-10% | 0-8% | 0-5% |
| Cu | 0-15% | 0-10% | 0-8% | 0-5% |
| Ir | 0-15% | 0-10% | 0-8% | 0-5% |
| Ti | 0-15% | 0-10% | 0-8% | 0-5% |
| Y | 0-15% | 0-10% | 0-8% | 0-5% |
| Zr | 0-15% | 0-10% | 0-8% | 0-5% |
| Ag | 0-15% | 0-10% | 0-8% | 0-5% |
| Al | 0-15% | 0-10% | 0-8% | 0-5% |
| Co | 0-15% | 0-10% | 0-8% | 0-5% |
| Fe | 0-15% | 0-10% | 0-8% | 0-5% |
| Mg | 0-15% | 0-10% | 0-8% | 0-5% |
| Ni | 0-15% | 0-10% | 0-8% | 0-5% |
| Pt | 0-15% | 0-10% | 0-8% | 0-5% |
| Si | 0-15% | 0-10% | 0-8% | 0-5% |
| Sn | 0-15% | 0-10% | 0-8% | 0-5% |
| Cs₂O | 0-1% | 0-1% | 0-1% | 0-1% |
| La₂O₃ | 0-1% | 0-1% | 0-1% | 0-1% |
| Y₂O₃ | 0-1% | 0-1% | 0-1% | 0-1% |
| ZrO₂ | 0-1% | 0-1% | 0-1% | 0-1% |
| C | <0.06 | <0.06 | <0.06 | <0.06 |
| N | <0.06 | <0.06 | <0.06 | <0.06 |
| O | <0.06 | <0.06 | <0.06 | <0.06 |

| | Wt. % | | | |
|---|---|---|---|---|
| Component | Ex. 111 | Ex. 112 | Ex. 113 | Ex. 114 |
| Mo | 40-95% | 40-95% | 40-95% | 40-95% |
| C | 0.01-0.3% | 0-0.3% | 0-0.3% | 0-0.3% |
| Co | ≤0.002% | ≤0.002% | ≤0.002% | ≤0.002% |
| Cs₂O | 0-0.2% | 0-0.2% | 0.01-0.2% | 0-0.2% |
| Fe | ≤0.02% | ≤0.02% | ≤0.02% | ≤0.02% |
| H | ≤0.002% | ≤0.002% | ≤0.002% | ≤0.002% |
| Hf | 0.1-2.5% | 0-2.5% | 0-2.5% | 0-2.5% |
| O | ≤0.06% | ≤0.06% | ≤0.06% | ≤0.06% |
| Os | ≤1% | ≤1% | ≤1% | ≤1% |
| La₂O₃ | 0-2% | 0.1-2% | 0-2% | 0-2% |
| N | ≤20 ppm | ≤20 ppm | ≤20 ppm | ≤20 ppm |
| Nb | ≤0.01% | ≤0.01% | ≤0.01% | ≤0.01% |
| Pt | ≤1% | ≤1% | ≤1% | ≤1% |
| Re | 5-40% | 5-40% | 5-40% | 5-40% |
| S | ≤0.008% | ≤0.008% | ≤0.008% | ≤0.008% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| W | 0-50% | 0-50% | 0-50% | 0.5-50% |
| Y₂O₃ | 0-1% | 0-1% | 0.1-1% | 0-1% |
| Zr | ≤1% | ≤1% | ≤1% | ≤1% |
| ZrO₂ | 0-3% | 0-3% | 0-3% | 0-3% |
| Ag | 0-5% | 0-5% | 0-5% | 0-5% |
| Al | 0-5% | 0-5% | 0-5% | 0-5% |
| Co | 0-5% | 0-5% | 0-5% | 0-5% |
| Mg | 0-5% | 0-5% | 0-5% | 0-5% |
| Ni | 0-5% | 0-5% | 0-5% | 0-5% |
| Si | 0-5% | 0-5% | 0-5% | 0-5% |
| Sn | 0-5% | 0-5% | 0-5% | 0-5% |
| CNT | 0-10% | 0-10% | 0-10% | 0-10% |

| | Wt. % | | |
|---|---|---|---|
| Component | Ex. 115 | Ex. 116 | Ex. 117 |
| Mo | 40-95% | 40-95% | 40-95% |
| C | 0-0.3% | 0-0.3% | 0-0.3% |
| Co | ≤0.002% | ≤0.002% | ≤0.002% |
| Cs₂O | 0-0.2% | 0-0.2% | 0-0.2% |
| H | ≤0.002% | ≤0.002% | ≤0.002% |
| Hf | 0-2.5% | 0-2.5% | 0-2.5% |
| O | ≤0.06% | ≤0.06% | ≤0.06% |
| Os | ≤1% | ≤1% | ≤1% |
| La₂O₃ | 0-2% | 0-2% | 0-2% |
| N | ≤20 ppm | ≤20 ppm | ≤20 ppm |
| Nb | ≤0.01% | ≤0.01% | ≤0.01% |
| Pt | ≤1% | ≤1% | ≤1% |
| Re | 5-40% | 5-40% | 5-40% |
| S | ≤0.008% | ≤0.008% | ≤0.008% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| W | 0-50% | 0-50% | 0-50% |
| Y₂O₃ | 0-1% | 0-1% | 0-1% |
| ZrO₂ | 0.1-3% | 0-3% | 0-3% |
| Ag | 0-5% | 0-5% | 0-5% |
| Al | 0-5% | 0-5% | 0-5% |
| Fe | 0-5% | 0-5% | 0-5% |
| Mg | 0-5% | 0-5% | 0-5% |
| Ni | 0-5% | 0-5% | 0-5% |
| Si | 0-5% | 0-5% | 0-5% |
| CNT | 0-10% | 0-10% | 0-10% |

| | Wt. % | | |
|---|---|---|---|
| Component | Ex. 118 | Ex. 119 | Ex. 120 |
| Mo | 60-95% | 60-95% | 60-90% |
| C | 0-0.3% | 0-0.3% | 0-0.3% |
| Co | ≤0.002% | ≤0.002% | ≤0.002% |
| Cs₂O | 0-0.2% | 0-0.2% | 0-0.2% |
| H | ≤0.002% | ≤0.002% | ≤0.002% |
| Hf | 0-2.5% | 0-2.5% | 0-2.5% |
| O | ≤0.06% | ≤0.06% | ≤0.06% |
| Os | ≤1% | ≤1% | ≤1% |
| La₂O₃ | 0-2% | 0-2% | 0-2% |
| N | ≤20 ppm | ≤20 ppm | ≤20 ppm |
| Nb | ≤0.01% | ≤0.01% | ≤0.01% |
| Pt | ≤1% | ≤1% | ≤1% |
| Re | 5-40% | 5-40% | 10-40% |
| S | ≤0.008% | ≤0.008% | ≤0.008% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| W | 0-50% | 0-50% | 0-50% |
| Y₂O₃ | 0-1% | 0-1% | 0-1% |
| ZrO₂ | 0.1-3% | 0-3% | 0-3% |
| Ag | 0-5% | 0-5% | 0-5% |
| Al | 0-5% | 0-5% | 0-5% |
| Fe | 0-5% | 0-5% | 0-5% |
| Mg | 0-5% | 0-5% | 0-5% |
| Ni | 0-5% | 0-5% | 0-5% |
| Si | 0-5% | 0-5% | 0-5% |
| CNT | 0-10% | 0-10% | 0-10% |

| | Wt. % | | | |
|---|---|---|---|---|
| Component | Ex. 121 | Ex. 122 | Ex. 123 | Ex. 124 |
| Mo | 60-95% | 60-95% | 50-95% | 40-80% |
| C | 0.05-0.15% | 0-0.15% | 0-0.15% | 0-0.15% |
| Cs₂O | 0-0.2% | 0-0.2% | 0.04-0.1% | 0-0.2% |
| Hf | 0.8-1.4% | 0-2% | 0-2.5% | 0-2.5% |
| La₂O₃ | 0-2% | 0.3-0.7% | 0-2% | 0-2% |
| Re | 5-40% | 5-40% | 5-40% | 5-40% |
| Ta | 0-2% | 0-2% | 0-50% | 0-50% |
| W | 0-2% | 0-2% | 0-50% | 20-50% |
| Y₂O₃ | 0-1% | 0-1% | 0.3-0.5% | 0-1% |
| ZrO₂ | 0-3% | 0-3% | 0-3% | 0-3% |

Wt. %

| Component | Ex. 125 | Ex. 126 | Ex. 127 |
|---|---|---|---|
| Mo | 97-95% | 50-90% | 60-95% |
| C | 0-0.15% | 0-0.15% | 0-0.15% |
| Cs₂O | 0-0.2% | 0-0.2% | 0-0.2% |
| Hf | 0-2.5% | 0-2.5% | 0-2.5% |
| La₂O₃ | 0-2% | 0-2% | 0-2% |
| Re | 5-30 | 5-40% | 5-40% |
| Ta | 0-3% | 10-50% | 0-40% |
| W | 0-3% | 0-50% | 0-40% |
| Y₂O₃ | 0-1% | 0-1% | 0-1% |
| ZrO₂ | 1.2-1.8% | 0-3% | 0-3% |

Wt. %

| Component | Ex. 128 | Ex. 129 | Ex. 130 |
|---|---|---|---|
| W | 20-95% | 60-95% | 20-80% |
| Re | 5-47.5% | 5-40% | 5-47.5% |
| Mo | 0-47.5% | <0.5% | 1-47.5% |
| Cu | <0.5% | <0.5% | <0.5% |
| C | ≤0.15% | ≤0.15% | ≤0.15% |
| Co | ≤0.002% | ≤0.002% | ≤0.002% |
| Cs₂O | ≤0.2% | ≤0.2% | ≤0.2% |
| Fe | ≤0.02% | ≤0.02% | ≤0.02% |
| H | ≤0.002% | ≤0.002% | ≤0.002% |
| Hf | <0.5% | <0.5% | <0.5% |
| La₂O₃ | <0.5% | <0.5% | <0.5% |
| O | ≤0.06% | ≤0.06% | ≤0.06% |
| Os | <0.5% | <0.5% | <0.5% |
| N | ≤20 ppm | ≤20 ppm | ≤20 ppm |
| Nb | <0.01% | ≤0.01% | ≤0.01% |
| Pt | <0.5% | <0.5% | <0.5% |
| S | ≤0.008% | ≤0.008% | ≤0.008% |
| Sn | ≤0.002% | <0.002% | ≤0.002% |
| Ta | <0.5% | <0.5% | <0.5% |
| Tc | <0.5% | <0.5% | <0.5% |
| Ti | <0.5% | <0.5% | <0.5% |
| V | <0.5% | <0.5% | <0.5% |
| Y₂O₃ | <0.5% | <0.5% | <0.5% |
| Zr | <0.5% | <0.5% | <0.5% |
| ZrO₂ | <0.5% | <0.5% | <0.5% |
| Ag | 0-5% | 0-5% | 0-5% |
| Al | 0-5% | 0-5% | 0-5% |
| Fe | 0-5% | 0-5% | 0-5% |
| Mg | 0-5% | 0-5% | 0-5% |
| Ni | 0-5% | 0-5% | 0-5% |
| Si | 0-5% | 0-5% | 0-5% |
| CNT | 0-10% | 0-10% | <0.5%. |

| | Wt. % | | | |
|---|---|---|---|---|
| Component | Ex. 131 | Ex. 132 | Ex. 133 | Ex. 134 |
| W | 1-94.9% | 1-94.9% | 1-94.9% | 10-95% |
| Cu | 0.1-94% | 0.1-94% | 0.1-94% | 1-84% |
| C | 0.01-0.3% | 0-0.3% | 0-0.3% | 0-0.3% |
| Co | ≤0.002% | ≤0.002% | ≤0.002% | ≤0.002% |
| Cs₂O | 0-0.2% | 0-0.2% | 0.01-0.2% | 0-0.2% |
| Fe | ≤0.02% | ≤0.02% | ≤0.02% | ≤0.02% |
| H | ≤0.002% | ≤0.002% | ≤0.002% | ≤0.002% |
| Hf | 0.1-2.5% | 0-2.5% | 0-2.5% | 0-2.5% |
| O | ≤0.06% | ≤0.06% | ≤0.06% | ≤0.06% |
| Os | ≤1% | ≤1% | ≤1% | ≤1% |
| La₂O₃ | 0-2% | 0.1-2% | 0-2% | 0-2% |
| Mo | 0-5% | 0.1-3% | 0-2% | 0-3% |
| N | ≤20 ppm | ≤20 ppm | ≤20 ppm | ≤20 ppm |
| Nb | ≤0.01% | ≤0.01% | ≤0.01% | ≤0.01% |
| Pt | ≤1% | ≤1% | ≤1% | ≤1% |
| Re | 5-40% | 5-40% | 5-40% | 6-40% |
| S | ≤0.008% | ≤0.008% | ≤0.008% | ≤0.008% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| Y₂O₃ | 0-1% | 0-1% | 0.1-1% | 0-1% |
| Zr | ≤1% | ≤1% | ≤1% | ≤1% |
| ZrO₂ | 0-3% | 0-3% | 0-3% | 0-3% |
| Ag | 0-5% | 0-5% | 0-5% | 0-5% |
| Al | 0-5% | 0-5% | 0-5% | 0-5% |
| Fe | 0-5% | 0-5% | 0-5% | 0-5% |
| Mg | 0-5% | 0-5% | 0-5% | 0-5% |
| Ni | 0-5% | 0-5% | 0-5% | 0-5% |
| Si | 0-5% | 0-5% | 0-5% | 0-5% |
| CNT | 0-10% | 0-10% | 0-10% | 0-10% |

| | Wt. % | | |
|---|---|---|---|
| Component | Ex. 135 | Ex. 136 | Ex. 137 |
| W | 20-96% | 25-92% | 30-88% |
| Cu | 2-74% | 2-68% | 5-62% |
| C | 0-0.3% | 0-0.3% | 0-0.3% |
| Co | ≤0.002% | ≤0.002% | ≤0.002% |
| Cs₂O | 0-0.2% | 0-0.2% | 0-0.2% |
| H | ≤0.002% | ≤0.002% | ≤0.002% |
| Hf | 0-2.5% | 0-2.5% | 0-2.5% |
| O | ≤0.06% | ≤0.06% | ≤0.06% |
| Os | ≤1% | ≤1% | ≤1% |
| La₂O₃ | 0-2% | 0-2% | 0-2% |
| Mo | 0-3% | 0-2% | 0-1% |
| N | ≤20 ppm | ≤20 ppm | ≤20 ppm |
| Nb | ≤0.01% | ≤0.01% | ≤0.01% |
| Pt | ≤1% | ≤1% | ≤1% |
| Re | 6-40% | 7-40% | 8-40% |
| S | ≤0.008% | ≤0.008% | ≤0.008% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| Y₂O₃ | 0-1% | 0-1% | 0-1% |
| ZrO₂ | 0.1-3% | 0-3% | 0-3% |
| Ag | 0-5% | 0-5% | 0-5% |
| Al | 0-5% | 0-5% | 0-5% |
| Fe | 0-5% | 0-5% | 0-5% |
| Mg | 0-5% | 0-5% | 0-5% |
| Ni | 0-5% | 0-5% | 0-5% |
| Si | 0-5% | 0-5% | 0-5% |
| CNT | 0-10% | 0-10% | 0-10% |

| | Wt. % | | | |
|---|---|---|---|---|
| Component | Ex. 138 | Ex. 139 | Ex. 140 | Ex. 141 |
| W | 25-88% | 35-87% | 40-86% | 50-85% |
| Cu | 5-68% | 5-57% | 5-51% | 5-40% |
| C | 0.05-0.15% | 0-0.15% | 0-0.15% | 0-0.15% |
| Cs₂O | 0-0.2% | 0-0.2% | 0.04-0.1% | 0-0.2% |
| Hf | 0.8-1.4% | 0-2.5% | 0-2.5% | 0-2.5% |
| La₂O₃ | 7-20% | 8-20% | 9-20% | 10-20% |
| Re | 0-40% | 0-40% | 0-40% | 0-40% |
| Ta | 0-50% | 0-50% | 0-50% | 0-50% |
| Y₂O₃ | 0-1% | 0-1% | 0.3-0.5% | 0-1% |
| ZrO₂ | 0-3% | 0-3% | 0-3% | 0-3% |

Wt. %

| Component | Ex. 142 | Ex. 143 | Ex. 144 |
|---|---|---|---|
| W | 55-88% | 60-87% | 70-86% |
| Cu | 1-34% | 1-28% | 1-17% |
| C | 0-0.15% | 0-0.15% | 0-0.15% |
| Cs₂O | 0-0.2% | 0-0.2% | 0-0.2% |
| Hf | 0-2.5% | 0-2.5% | 0-2.5% |
| La₂O₃ | 0-2% | 0-2% | 0-2% |
| Re | 11-40% | 12-40% | 13-40% |
| Ta | 0-50% | 10-50% | 0-50% |
| W | 0-50% | 0-50% | 0-50% |
| Y₂O₃ | 0-1% | 0-1% | 0-1% |
| ZrO₂ | 1.2-1.8% | 0-3% | 0-3% |

| | Wt. % | | |
|---|---|---|---|
| Component | Ex. 145 | Ex. 146 | Ex. 147 |
| Ti | 55-66% | 65-76% | 70-76% |
| Mo | 20-41% | 20-31% | 20-26% |
| Re | 4-20% | 4-20% | 4-20% |
| Yt | <0.5% | <0.5% | <0.5% |
| Nb | <0.5% | <0.5% | <0.5% |
| Co | <0.5% | <0.5% | <0.5% |
| Cr | <0.5% | <0.5% | <0.5% |
| Zr | <0.5% | <0.5% | <0.5% |
| C | ≤0.15% | ≤0.15% | ≤0.15% |
| O | ≤0.06% | ≤0.06% | ≤0.06% |
| N | ≤20 ppm | ≤20 ppm | ≤20 ppm |

| | Wt. % | | |
|---|---|---|---|
| Component | Ex. 148 | Ex. 149 | Ex. 150 |
| W | 20-95% | 60-85% | 20-84% |
| Re | 5-47.5% | 15-40% | 5-47.5% |
| Mo | 0-47.5% | <0.5% | 1-47.5% |

Wt. %

| Component | Ex. 151 | Ex. 152 | Ex. 153 |
|---|---|---|---|
| W | 50.1-93% | 65-92% | 70-90% |
| Re | 7-40% | 8-35% | 9-30% |
| Mo | 0-40% | <0.5% | 1-30% |

| | Wt. % | | |
|---|---|---|---|
| Component | Ex. 154 | Ex. 155 | Ex. 156 |
| W | 20-49% | 20-49% | 20-49% |
| Re | 5-40% | 5-40% | 5-39% |
| Mo | 20-60% | 30-60% | 40-60% |

| | Wt. % | | |
|---|---|---|---|
| Component | Ex. 157 | Ex. 158 | Ex. 159 |
| W | 20-40% | 20-35% | 20-30% |
| Re | 7-40% | 10-40% | 25-40% |
| Mo | 0-40% | 10-40% | 25-40% |

Wt. %

| Component | Ex. 160 | Ex. 161 | Ex. 162 |
|---|---|---|---|
| W | 20-95% | 60-93% | 20-80% |
| Re | 5-47.5% | 7-40% | 5-47.5% |
| Mo | 0-47.5% | <0.5% | 1-47.5% |
| Cu | <0.5% | <0.5% | <0.5% |
| C | ≤0.15% | ≤0.15% | ≤0.15% |
| Co | ≤0.002% | ≤0.002% | ≤0.002% |
| Cs₂O | ≤0.2% | ≤0.2% | ≤0.2% |
| Fe | ≤0.02% | ≤0.02% | ≤0.02% |
| H | ≤0.002% | ≤0.002% | ≤0.002% |
| Hf | <0.5% | <0.5% | <0.5% |
| La₂O₃ | <0.5% | <0.5% | <0.5% |
| O | ≤0.06% | ≤0.06% | ≤0.06% |
| Os | <0.5% | <0.5% | <0.5% |
| N | ≤20 ppm | ≤20 ppm | ≤20 ppm |
| Nb | ≤0.01% | ≤0.01% | ≤0.01% |
| Pt | <0.5% | <0.5% | <0.5% |
| S | ≤0.008% | ≤0.008% | ≤0.008% |
| Sn | ≤0.002% | ≤0.002% | ≤0.002% |
| Ta | <0.5% | <0.5% | <0.5% |
| Tc | <0.5% | <0.5% | <0.5% |
| Ti | <0.5% | <0.5% | <0.5% |
| V | <0.5% | <0.5% | <0.5% |
| Y₂O₃ | <0.5% | <0.5% | <0.5% |
| Zr | <0.5% | <0.5% | <0.5% |
| ZrO₂ | <0.5% | <0.5% | <0.5% |
| Ag | 0-5% | 0-5% | 0-5% |
| Al | 0-5% | 0-5% | 0-5% |
| Fe | 0-5% | 0-5% | 0-5% |
| Mg | 0-5% | 0-5% | 0-5% |
| Ni | 0-5% | 0-5% | 0-5% |
| Si | 0-5% | 0-5% | 0-5% |
| CNT | 0-10% | 0-10% | <0.5% |

| | Wt. % | | | |
|---|---|---|---|---|
| Component | Ex. 163 | Ex. 164 | Ex. 165 | Ex. 166 |
| Ag | 0-10% | 0-10% | 0-10% | 0-10% |
| Al | 0-10% | 0-10% | 0-10% | 2-10% |
| B | 0-10% | 0-10% | 0-10% | 0-10% |
| Bi | 0-10% | 0-10% | 0-10% | 0-10% |
| Cr | 2-30% | 10-30% | 0-20% | 0-20% |
| Cu | 0-10% | 0-10% | 0-10% | 0-10% |
| Co | 0-10% | 32-70% | 0-10% | 0-10% |
| Fe | 50-80% | 0-20% | 0-10% | 0-10% |
| Hf | 0-10% | 0-10% | 0-10% | 0-10% |
| Ir | 0-10% | 0-10% | 0-10% | 0-10% |
| La | 0-10% | 0-10% | 0-10% | 0-10% |
| Mg | 0-10% | 0-10% | 0-10% | 0-10% |
| Mn | 0-20% | 0-10% | 0-10% | 0-10% |
| Mo | 0-10% | 0-30% | 0-16% | 0-16% |
| Nb | 0-10% | 0-10% | 0-10% | 0-10% |
| Ni | 0.1-30% | 0.1-40% | 0-10% | 0-10% |
| Os | 0-10% | 0-10% | 0-10% | 0-10% |
| Pt | 0-10% | 0-10% | 0-10% | 0-10% |
| Re | 5-40% | 4.8-40% | 4.5-80% | 4.5-80% |
| Rh | 0-10% | 0-10% | 0-10% | 0-10% |
| Se | 0-10% | 0-10% | 0-10% | 0-10% |
| Si | 0-10% | 0-10% | 0-10% | 0-10% |
| Sn | 0-10% | 0-10% | 0-12% | 0-12% |
| Ta | 0-10% | 0-10% | 0-10% | 0-10% |
| Tc | 0-10% | 0-10% | 0-10% | 0-10% |
| Ti | 0-10% | 0-10% | 70-91.5% | 70-91.5% |
| V | 0-10% | 0-10% | 0-10% | 0.01-10% |
| W | 0-10% | 0-20% | 0-10% | 0-10% |
| Y | 0-10% | 0-10% | 0-10% | 0-10% |
| Zr | 0-10% | 0-10% | 0-10% | 0-10% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06 | <0.06 | <0.06 | <0.06 |
| N | <0.06 | <0.06 | <0.06 | <0.06 |
| O | <0.06 | <0.06 | <0.06 | <0.06 |

| | Wt. % | | | |
|---|---|---|---|---|
| Component | Ex. 167 | Ex. 168 | Ex. 169 | Ex. 170 |
| Ag | 0-10% | 0-10% | 0-10% | 0-10% |
| Al | 0-10% | 0-10% | 0-10% | 0-10% |
| B | 0-10% | 0-10% | 0-10% | 0-10% |
| Bi | 0-10% | 0-10% | 0-10% | 0-10% |
| Cr | 0-10% | 0-20% | 0-20% | 0-10% |
| Cu | 0-10% | 0-10% | 0-50% | 0-10% |
| Co | 0-10% | 0-10% | 0-10% | 0-10% |
| Fe | 0-10% | 0-10% | 0-10% | 0-10% |
| Hf | 0-10% | 0-10% | 0-10% | 0-10% |
| Ir | 0-10% | 0-10% | 0-10% | 0-12% |
| La | 0-10% | 0-10% | 0-10% | 0-10% |
| Mg | 0-10% | 0-10% | 0-10% | 0-10% |
| Mn | 0-10% | 0-10% | 0-10% | 0-10% |
| Mo | 0-55% | 40-93% | 0-50% | 0-20% |
| Nb | 0-10% | 0-10% | 0-10% | 40-85% |
| Ni | 0-45% | 0-10% | 0-10% | 0-10% |
| Os | 0-10% | 0-10% | 0-10% | 0-10% |
| Pt | 0-10% | 0-10% | 0-10% | 0-10% |
| Re | 14-40% | 7-40% | 7-40% | 7-40% |
| Rh | 0-10% | 0-10% | 0-10% | 0-10% |
| Se | 0-10% | 0-10% | 0-10% | 0-10% |
| Si | 0-10% | 0-10% | 0-10% | 0-10% |
| Sn | 0-10% | 0-10% | 0-10% | 0-10% |
| Ta | 35-84% | 0-50% | 0-50% | 0-35% |
| Tc | 0-10% | 0-10% | 0-10% | 0-10% |
| Ti | 0-10% | 0-10% | 0-10% | 0-10% |
| V | 0-10% | 0-10% | 0-10% | 0-10% |
| W | 0.1-25% | 0-50% | 14-10% | 0-15% |
| Y | 0-10% | 0-10% | 0-10% | 0-10% |
| Zr | 0-10% | 0-10% | 0-50% | 0-10% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06 | <0.06 | <0.06 | <0.06 |
| N | <0.06 | <0.06 | <0.06 | <0.06 |
| O | <0.06 | <0.06 | <0.06 | <0.06 |

| | Wt. % | | | |
|---|---|---|---|---|
| Component | Ex. 171 | Ex. 172 | Ex. 173 | Ex. 174 |
| Ag | 0-10% | 0-10% | 0-5% | 0-5% |
| Al | 0-10% | 0-10% | 0-5% | 5-7% |
| B | 0-10% | 0-10% | 0-5% | 0-5% |
| Bi | 0-10% | 0-10% | 0-5% | 0-5% |
| Cr | 0-10% | 1-95% | 12-28% | 0-5% |
| Cu | 0-10% | 0-10% | 0-5% | 0-5% |
| Co | 0-10% | 0-10% | 36-68% | 0-5% |
| Fe | 0-10% | 0-10% | 0-18% | 0-5% |
| Hf | 0-10% | 0-10% | 0-5% | 0-5% |
| Ir | 0-10% | 0-10% | 0-5% | 0-5% |
| La | 0-10% | 0-10% | 0-5% | 0-5% |
| Mg | 0-10% | 0-10% | 0-5% | 0-5% |
| Mn | 0-10% | 0-10% | 0-5% | 0-5% |
| Mo | 0-10% | 0-20% | 0-12% | 0-5% |
| Nb | 0-10% | 0-10% | 0-5% | 0-5% |
| Ni | 30-58% | 0-10% | 9-36% | 0-5% |
| Os | 0-10% | 0-10% | 0-5% | 0-5% |
| Pt | 0-10% | 0-10% | 0-5% | 0-5% |
| Re | 5-40% | 5-40% | 4.8-40% | 4.5-40% |
| Rh | 0-10% | 0-10% | 0-5% | 0-5% |
| Se | 0-10% | 0-10% | 0-5% | 0-5% |
| Si | 0-10% | 0-10% | 0-5% | 0-5% |
| Sn | 0-10% | 0-10% | 0-5% | 0-5% |
| Ta | 0-10% | 0-10% | 0-5% | 0-5% |
| Tc | 0-10% | 0-10% | 0-5% | 0-5% |
| Ti | 30-58% | 0-40% | 0-5% | 70-91.5% |
| V | 0-10% | 0-10% | 0-5% | 3-6% |
| W | 0-10% | 0-10% | 0-16% | 0-5% |
| Y | 0-10% | 0-10% | 0-5% | 0-5% |
| Zr | 0-10% | 0-20% | 0-5% | 0-5% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06 | <0.06 | <0.06 | <0.06 |
| N | <0.06 | <0.06 | <0.06 | <0.06 |
| O | <0.06 | <0.06 | <0.06 | <0.06 |

| Compo- | Wt. % | | | |
|---|---|---|---|---|
| nent | Ex. 175 | Ex. 176 | Ex. 177 | Ex. 178 |
| Ag | 0-8% | 0-8% | 0-8% | 0-8% |
| Al | 0-8% | 0-8% | 0-8% | 2-10% |
| B | 0-8% | 0-8% | 0-8% | 0-8% |
| Bi | 0-8% | 0-8% | 0-8% | 0-8% |
| Cr | 2-30% | 10-30% | 0-20% | 0-20% |
| Cu | 0-8% | 0-8% | 0-8% | 0-8% |
| Co | 0-8% | 32-70% | 0-8% | 0-8% |
| Fe | 50-80% | 0-20% | 0-8% | 0-8% |
| Hf | 0-8% | 0-8% | 0-8% | 0-8% |
| Ir | 0-8% | 0-8% | 0-8% | 0-8% |
| La | 0-8% | 0-8% | 0-8% | 0-8% |
| Mg | 0-8% | 0-8% | 0-8% | 0-8% |
| Mn | 0-20% | 0-8% | 0-8% | 0-8% |
| Mo | 0-8% | 0-30% | 0-16% | 0-16% |
| Nb | 0-8% | 0-8% | 0-8% | 0-8% |
| Ni | 0.1-30% | 0.1-40% | 0-8% | 0-8% |
| Os | 0-8% | 0-8% | 0-8% | 0-8% |
| Pt | 0-8% | 0-8% | 0-8% | 0-8% |
| Re | 5-40% | 4.8-40% | 4.5-80% | 4.5-80% |
| Rh | 0-8% | 0-8% | 0-8% | 0-8% |
| Se | 0-8% | 0-8% | 0-8% | 0-8% |
| Si | 0-8% | 0-8% | 0-8% | 0-8% |
| Sn | 0-8% | 0-8% | 0-12% | 0-12% |
| Ta | 0-8% | 0-8% | 0-8% | 0-8% |
| Tc | 0-8% | 0-8% | 0-8% | 0-8% |
| Ti | 0-8% | 0-8% | 70-91.5% | 70-91.5% |
| V | 0-8% | 0-8% | 0-8% | 0.01-10% |
| W | 0-8% | 0-20% | 0-8% | 0-8% |
| Y | 0-8% | 0-8% | 0-8% | 0-8% |
| Zr | 0-8% | 0-8% | 0-8% | 0-8% |

| | Wt. % | | | |
|---|---|---|---|---|
| Component | Ex. 179 | Ex. 180 | Ex. 181 | Ex. 182 |
| Ag | 0-8% | 0-8% | 0-8% | 0-8% |
| Al | 0-8% | 0-8% | 0-8% | 0-8% |
| B | 0-8% | 0-8% | 0-8% | 0-8% |
| Bi | 0-8% | 0-8% | 0-8% | 0-8% |
| Cr | 0-8% | 0-20% | 0-20% | 0-8% |
| Cu | 0-8% | 0-8% | 0-50% | 0-8% |
| Co | 0-8% | 0-8% | 0-8% | 0-8% |
| Fe | 0-8% | 0-8% | 0-8% | 0-8% |
| Hf | 0-8% | 0-8% | 0-8% | 0-8% |
| Ir | 0-8% | 0-8% | 0-8% | 0-12% |
| La | 0-8% | 0-8% | 0-8% | 0-8% |
| Mg | 0-8% | 0-8% | 0-8% | 0-8% |
| Mn | 0-8% | 0-8% | 0-8% | 0-8% |
| Mo | 0-55% | 40-93% | 0-50% | 0-20% |
| Nb | 0-8% | 0-8% | 0-8% | 40-85% |
| Ni | 0-45% | 0-8% | 0-8% | 0-8% |
| Os | 0-8% | 0-8% | 0-8% | 0-8% |
| Pt | 0-8% | 0-8% | 0-8% | 0-8% |
| Re | 14-40% | 7-40% | 7-40% | 7-40% |
| Rh | 0-8% | 0-8% | 0-8% | 0-8% |
| Se | 0-8% | 0-8% | 0-8% | 0-8% |
| Si | 0-8% | 0-8% | 0-8% | 0-8% |
| Sn | 0-8% | 0-8% | 0-8% | 0-8% |
| Ta | 35-84% | 0-50% | 0-50% | 0-35% |
| Tc | 0-8% | 0-8% | 0-8% | 0-8% |
| Ti | 0-8% | 0-8% | 0-8% | 0-8% |
| V | 0-8% | 0-8% | 0-8% | 0-8% |
| W | 0.1-25% | 0-50% | 14-10% | 0-15% |
| Y | 0-8% | 0-8% | 0-8% | 0-8% |
| Zr | 0-8% | 0-8% | 0-50% | 0-8% |

| | Wt. % | | | |
|---|---|---|---|---|
| Component | Ex. 183 | Ex. 184 | Ex. 185 | Ex. 186 |
| Ag | 0-5% | 0-5% | 0-5% | 0-5% |
| Al | 0-5% | 0-5% | 0-5% | 5-7% |
| B | 0-5% | 0-5% | 0-5% | 0-5% |
| Bi | 0-5% | 0-5% | 0-5% | 0-5% |
| Cr | 0-5% | 1-95% | 12-28% | 0-5% |
| Cu | 0-5% | 0-5% | 0-5% | 0-5% |
| Co | 0-5% | 0-5% | 36-68% | 0-5% |
| Fe | 0-5% | 0-5% | 0-18% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 0-20% | 0-12% | 0-5% |
| Nb | 0-5% | 0-5% | 0-5% | 0-5% |
| Ni | 30-58% | 0-5% | 9-36% | 0-5% |
| Os | 0-5% | 0-5% | 0-5% | 0-5% |
| Pt | 0-5% | 0-5% | 0-5% | 0-5% |
| Re | 5-40% | 5-40% | 4.8-40% | 4.5-40% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 0-5% |
| Tc | 0-5% | 0-5% | 0-5% | 0-5% |
| Ti | 30-58% | 0-40% | 0-5% | 70-91.5% |
| V | 0-5% | 0-5% | 0-5% | 3-6% |
| W | 0-5% | 0-5% | 0-16% | 0-5% |
| Y | 0-5% | 0-5% | 0-5% | 0-5% |
| Zr | 0-5% | 0-20% | 0-5% | 0-5% |

| | Wt. % | | | |
|---|---|---|---|---|
| Component | Ex. 187 | Ex. 188 | Ex. 189 | Ex. 190 |
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 1-15% | 2-10% | 3-8% | 0-5% |
| Nb | 0-5% | 0-5% | 0-5% | 20-45% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 1-15% |
| Tc | 0-5% | 0-5% | 0-5% | 0-5% |
| Ti | 51-70% | 51-70% | 55-70% | 51-70% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 20-40% | 22-38% | 27-33% | 1-15% |

| | Wt. % | | | |
|---|---|---|---|---|
| Component | Ex. 191 | Ex. 192 | Ex. 193 | Ex. 194 |
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 0-5% | 0-5% | 0-5% |
| Nb | 25-40% | 30-40% | 25-40% | 26-32% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 2-8% | 3-6% | 5-15% | 10-14% |
| Tc | 0-5% | 0-5% | 0-5% | 0-5% |
| Ti | 51-70% | 52-63% | 51-68% | 51-62% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 2-12% | 4-8% | 2-8% | 2-6% |

| | Wt. % | | | |
|---|---|---|---|---|
| Component | Ex. 195 | Ex. 196 | Ex. 197 | Ex. 198 |
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 5-35% | 10-30% | 15-25% |
| Cu | 0-5% | 0-5% | 0-5% | 0-5% |
| Co | 0-5% | 20-55% | 25-50% | 35-45% |
| Fe | 0-5% | 3-25% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 2-15% | 3-12% | 4-9% |
| Nb | 30-40% | 0-5% | 0-5% | 0-5% |
| Ni | 0-5% | 4-23% | 5-20% | 10-18% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 1-3% | 0-5% | 0-5% | 0-5% |
| Tc | 0-5% | 0-5% | 0-5% | 0-5% |
| Ti | 51-67% | 0-5% | 0-5% | 0-5% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 2-5% | 0-5% | 0-5% | 0-5% |

| | Wt. % | | | |
|---|---|---|---|---|
| Component | Ex. 199 | Ex. 200 | Ex. 201 | Ex. 202 |
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 30-65% | 40-60% | 45-55% | 0-5% |
| Nb | 0-5% | 0-5% | 0-5% | 55-99.75% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 0-5% |
| Tc | 0-5% | 0-5% | 0-5% | 0-5% |
| Ti | 0-5% | 0-5% | 0-5% | 0-5% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 30-56% | 40-60% | 45-55% | 0.25-45% |

| | Wt. % | | | |
|---|---|---|---|---|
| Component | Ex. 203 | Ex. 204 | Ex. 205 | Ex. 206 |
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 0-5% | 0-5% | 0-5% |
| Nb | 75-99.5% | 95-99.25% | 55-78.5% | 68-74.25% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 20-35% | 25-30% |
| Tc | 0-5% | 0-5% | 0-5% | 0-5% |
| Ti | 0-5% | 0-5% | 0-5% | 0-5% |
| V | 0-5% | 0-5% | 0-5% | 0-5% |
| W | 0-5% | 0-5% | 1-8% | 0-5% |
| Y | 0-5% | 0-5% | 0-5% | 0-5% |
| Zr | 0.5-25% | 0.75-5% | 0.5-5% | 0.75-3% |

Wt. %

| Element | Ex. 207 | Ex. 208 | Ex. 209 | Ex. 210 |
|---|---|---|---|---|
| Re | 30-75% | 40-75% | 45-75% | 45-70% |
| Cr | 25-70% | 25-65% | 25-55% | 30-55% |
| Mo | 0-25% | 0-25% | 1-25% | 2-25% |
| Bi | 0-25% | 0-25% | 0-25% | 0-25% |
| Cr | 0-25% | 0-25% | 0-25% | 0-25% |
| Ir | 0-25% | 0-25% | 0-25% | 0-25% |
| Nb | 0-25% | 0-25% | 0-25% | 0-25% |
| Ta | 0-25% | 0-25% | 0-25% | 0-25% |
| V | 0-25% | 0-25% | 0-25% | 0-25% |
| W | 0-25% | 0-25% | 0-25% | 0-25% |
| Mn | 0-25% | 0-25% | 0-25% | 0-25% |
| Tc | 0-25% | 0-25% | 0-25% | 0-25% |
| Ru | 0-25% | 0-25% | 0-25% | 0-25% |
| Rh | 0-25% | 0-25% | 0-25% | 0-25% |
| Hf | 0-25% | 0-25% | 0-25% | 0-25% |
| Os | 0-25% | 0-25% | 0-25% | 0-25% |
| Cu | 0-25% | 0-25% | 0-25% | 0-25% |
| Ir | 0-25% | 0-25% | 0-25% | 0-25% |
| Ti | 0-25% | 0-25% | 0-25% | 0-25% |
| Y | 0-25% | 0-25% | 0-25% | 0-25% |
| Zr | 0-25% | 0-25% | 0-25% | 0-25% |
| C | <0.06 | <0.06 | <0.06 | <0.06 |
| N | <0.06 | <0.06 | <0.06 | <0.06 |
| O | <0.06 | <0.06 | <0.06 | <0.06 |

In Examples 1-210, it will be appreciated that all of the above ranges include any value between the range and any other range that is between the ranges set forth above. Any of the above values that include the ≤symbol includes the range from 0 to the stated value and all values and ranges therebetween.

In accordance with another and/or alternative non-limiting aspect of the present disclosure, the medical device is generally designed to include at least about 5 wt. % of the metal alloy (e.g., 5-100 wt. % and all values and ranges therebetween). In one non-limiting embodiment of the disclosure, the medical device includes at least about 50 wt. % of the metal alloy. In another non-limiting embodiment of the disclosure, the medical device includes at least about 95 wt. % of the metal alloy. In one specific configuration, when the medical device includes an expandable frame, the expandable frame is formed of 50-100 wt. % (and all values and ranges therebetween) of the metal alloy, and typically 75-100 wt. % of the metal alloy.

In accordance with another and/or alternative non-limiting aspect of the present disclosure, there is provided a medical device partially or fully formed of metal alloy that includes rhenium in a sufficient quantity as to create a “rhenium effect” in the metal alloy. As defined herein, a “rhenium effect” is a) an increase of at least 10% in ductility of the metal alloy caused by the addition of rhenium to the metal alloy, and/or b) an increase of at least 10% in tensile strength of the metal alloy caused by the addition of rhenium to the metal alloy. It has been found for many metal alloys results in improved ductility and/or tensile strength. It has been found that the addition of rhenium to a metal alloy can result in the formation of a twining alloy in the metal alloy that results in the overall ductility of the metal alloy to increase as the yield and tensile strength increases as a result of reduction and/or work hardening of the metal alloy that includes the rhenium addition. The “rhenium effect” occurs when the atomic weight of rhenium in the metal alloy is at least 15% (e.g., 15 awt. % to 99 awt. % rhenium in the metal alloy and all values and ranges therebetween). For example, for standard stainless steel alloys, the “rhenium effect” can begin to be present when the stainless steel alloy is modified to include a rhenium amount of at least 5-10 wt. % (and all values and ranges therebetween) of the stainless steel alloy. For standard CoCr alloys, the “rhenium effect” can begin to be present when the CoCr alloy is modified to include a rhenium amount of at least 4.8-9.5 wt. % (and all values and ranges therebetween) of the CoCr alloy. For standard TiAlV alloys, the “rhenium effect” can begin to be present when the TiAlV alloy is modified to include a rhenium amount of at least 4.5-9 wt. % (and all values and ranges therebetween) of the TiAlV alloy. At can be appreciated, the rhenium content in the above examples can be greater than the minimum amount to create the “rhenium effect” in the metal alloy.

In accordance with another and/or alternative non-limiting aspect of the present disclosure, there is provided a medical device that is formed of 50-100% (and all values and ranges therebetween) of a metal alloy that includes rhenium in a sufficient amount to create a “rhenium effect” in the metal alloy. In one non-limiting embodiment, the metal alloy includes at least 15 awt. % rhenium (e.g., 15-99.9 awt. % and all values and ranges therebetween), and at least 0.1 wt. % (e.g., 0.1 wt. % to 96 wt. % an all values and ranges therebetween) of one or more additives selected from the group of aluminum, bismuth, calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, and/or zirconium oxide. In another non-limiting embodiment, the metal alloy includes at least 15 awt. % rhenium (e.g., 15-99.9 awt. % and all values and ranges therebetween), and at least 0.1 wt. % (e.g., 0.1 wt. % to 96 wt. % an all values and ranges therebetween) of two or more additives selected from the group of aluminum, bismuth, calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, and/or zirconium oxide. In another non-limiting embodiment, the metal alloy includes at least 15 awt. % rhenium (e.g., 15-99.9 awt. % and all values and ranges therebetween), and at least 0.1 wt. % (e.g., 0.1 wt. % to 96 wt. % an all values and ranges therebetween) of three or more additives selected from the group of aluminum, bismuth, calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, and/or zirconium oxide.

In another and/or alternative non-limiting aspect of the present disclosure, the metal alloy optionally includes less than about 5 wt. % (e.g., 0-4.999999 wt. % and all values and ranges therebetween) other metals and/or impurities, typically 0-1 wt. %, more typically 0-0.1 wt. %, even more typically 0-0.01 wt. %, and still even more typically 0-0.001 wt. %. A high purity level of the metal alloy results in the formation of a more homogeneous alloy, which in turn results in a more uniform density throughout the metal alloy, and also results in the desired yield and ultimate tensile strengths of the metal alloy.

In accordance with another and/or alternative non-limiting aspect of the present disclosure, at least 30 wt. % (e.g., 30-100 wt. % and all values and ranges therebetween) of the metal alloy includes one or more of molybdenum, niobium, rhenium, tantalum, or tungsten. In another non-limiting embodiment, at least 40 wt. % of the metal alloy includes one or more of molybdenum, niobium, rhenium, tantalum, or tungsten. In another non-limiting embodiment, at least 50 wt. % of the metal alloy includes one or more of molybdenum, niobium, rhenium, tantalum, or tungsten.

In another non-limiting embodiment, at least 50 wt. % (e.g., 50-100 wt. % and all values and ranges therebetween) of the metal alloy includes one or more of molybdenum, niobium, rhenium, tantalum, titanium, zirconium or tungsten, and 1-40 wt. % (and all values and ranges therebetween) of the metal alloy includes one or more additives selected from the group of aluminum, bismuth, calcium, carbon, cerium oxide, chromium, cobalt, copper, gold, hafnium, iridium, iron, lanthanum, lanthanum oxide, lead, magnesium, manganese, nickel, osmium, platinum, rare earth metals, rhodium, ruthenium, silver, technetium, vanadium, yttrium, yttrium oxide, zinc, and/or zirconium oxide.

In accordance with another and/or alternative non-limiting aspect of the present disclosure, there is provided a metal alloy wherein at least 20 wt. % (e.g., 20-99 wt. % and all values and ranges therebetween) of the metal alloy includes rhenium. In one non-limiting embodiment, the metal alloy includes at least 20 wt. % (e.g., 20-99.9 wt. % and all values and ranges therebetween) rhenium, and 0.1-80 wt. % (and all values and ranges therebetween) of one or more additives selected from the group of aluminum, bismuth, calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, and/or zirconium oxide.

In another non-limiting aspect of the present disclosure, the metals used to form the metal alloy includes rhenium and tungsten and optionally one or more alloying agents such as, but not limited to, aluminum, bismuth, calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, vanadium, yttrium, yttrium oxide, zinc, zirconium, and/or zirconium oxide, and/or alloys of one or more of such components (e.g., WRe, WReMo, etc.). In one non-limiting formulation, the metal alloy includes up to 40 wt. % rhenium and at least 60 wt. % tungsten. In one non-limiting embodiment, the total weight percent of the tungsten and rhenium in the tungsten-rhenium alloy is at least about 95 wt. %, typically at least about 99 wt. %, more typically at least about 99.5 wt. %, yet more typically at least about 99.9 wt. %, and still more typically at least about 99.99 wt. %. In another non-limiting formulation, the metal alloy includes up to 47.5 wt. % rhenium and at least 20-80 wt. % tungsten (and all values and ranges therebetween) and 1-47.5 wt. % molybdenum (and all values and ranges therebetween).

In accordance with another and/or alternative non-limiting aspect of the present disclosure, at least 35 wt. % (e.g., 35-75 wt. % and all values and ranges therebetween) of the metal alloy includes rhenium, and the metal alloy also includes chromium. In one non-limiting embodiment, at least 25 wt. % (e.g., 25-49.9 wt. % and all values and ranges therebetween) of the metal alloy includes chromium. In another non-limiting embodiment, at least 30 wt. % of the metal alloy includes chromium. In another non-limiting embodiment, at least 33 wt. % of the metal alloy includes chromium. In another non-limiting embodiment, at least 50 wt. % (e.g., 50-74.9 wt. % and all values and ranges therebetween) of the metal alloy includes rhenium, at least 25 wt. % (e.g., 25-49.9 wt. % and all values and ranges therebetween) of the metal alloy includes chromium, and 0.1-25 wt. % (and all values and ranges therebetween) of the metal alloy includes one or more of molybdenum, bismuth, niobium, tantalum, titanium, vanadium, tungsten, manganese, zirconium, technetium, ruthenium, rhodium, hafnium, osmium, copper, yttrium, zirconium, and/or iridium. In another non-limiting embodiment, at least 55 wt. % (e.g., 55-69.9 wt. % and all values and ranges therebetween) of the metal alloy includes rhenium, at least 30 wt. % (e.g., 30-44.9 wt. % and all values and ranges therebetween) of the metal alloy includes chromium, and 0.1-15 wt. % (and all values and ranges therebetween) of the metal alloy includes one or more of molybdenum, bismuth, niobium, tantalum, titanium, vanadium, tungsten, manganese, zirconium, technetium, ruthenium, rhodium, hafnium, osmium, copper, yttrium, zirconium, and/or iridium.

In accordance with another and/or alternative non-limiting aspect of the present disclosure, metal alloy includes 10-60 atomic weight percent (awt. %) Re (and all values and ranges therebetween) and one or more metals selected from the group consisting of Mo, Cr, Ta, Nb, Ti, and Zr. In one non-limiting embodiment, the metal alloy includes 15-60 atw % Re and one or more metals selected from the group consisting of Cr, Ta, Nb, Ti, and Zr. In another non-limiting embodiment, the metal alloy includes 15-60 atw % Re and one or more metals selected from the group consisting of 0.5-70 awt. % Cr (and all values and ranges therebetween), 0.5-70 awt. % Ta (and all values and ranges therebetween), 0.5-70 at. % Nb (and all values and ranges therebetween), 0.5-70 awt. % Ti (and all values and ranges therebetween), and 0.5-70 awt. % Zr (and all values and ranges therebetween).

In accordance with another and/or alternative non-limiting aspect of the present disclosure, the metal alloy includes 0.5-50 awt. % Re (and all values and ranges therebetween) and 0.5-70 awt. % Cr (and all values and ranges therebetween).

In accordance with another and/or alternative non-limiting aspect of the present disclosure, the metal alloy includes 0.5-50 awt. % Re (and all values and ranges therebetween) and 0.5-70 awt. % Ta (and all values and ranges therebetween).

In accordance with another and/or alternative non-limiting aspect of the present disclosure, the metal alloy includes 0.5-50 awt. % Re (and all values and ranges therebetween) and 0.5-70 awt. % Nb (and all values and ranges therebetween).

In accordance with another and/or alternative non-limiting aspect of the present disclosure, the metal alloy includes 0.5-50 awt. % Re (and all values and ranges therebetween) and 0.5-70 awt. % Ti (and all values and ranges therebetween).

In accordance with another and/or alternative non-limiting aspect of the present disclosure, the metal alloy includes greater than 50 wt. % titanium (e.g., 51-80 wt. % and all values and ranges therebetween), 15-45 wt. % (and all values and ranges therebetween) niobium, 1-10 wt. % (and all values and ranges therebetween) zirconium, and 1-15 wt. % (and all values and ranges therebetween) tantalum. In one non-limiting formulation, the metal alloy includes 58-70 wt. % titanium, 27-37 wt. % niobium, and 2-9 wt. % zirconium, and 1-15 wt. % tantalum.

In accordance with another and/or alternative non-limiting aspect of the present disclosure, the metal alloy includes greater than 50 wt. % titanium (e.g., 51-80 wt. % and all values and ranges therebetween), 15-45 wt. % (and all values and ranges therebetween) niobium, and 1-10 wt. % (and all values and ranges therebetween) molybdenum. In one non-limiting formulation, the metal alloy includes 58-69 wt. % titanium, 27-33 wt. % niobium, and 4-8 wt. % molybdenum.

In accordance with another and/or alternative non-limiting aspect of the present disclosure, the metal alloy includes 30-60 wt. % cobalt (and all values and ranges therebetween), 10-30 wt. % chromium (and all values and ranges therebetween), 5-20 wt. % iron (and all values and ranges therebetween), 5-22 wt. % nickel (and all values and ranges therebetween), and 2-12 wt. % molybdenum (and all values and ranges therebetween). In one non-limiting formulation, the metal alloy includes 35-45 wt. % cobalt, 15-25 wt. % chromium, 12-20 wt. % iron, 10-20 wt. % nickel, and 5-9 wt. % molybdenum.

In accordance with another and/or alternative non-limiting aspect of the present disclosure, the metal alloy includes 40-60 wt. % zirconium (and all values and ranges therebetween), and 40-60 wt. % molybdenum (and all values and ranges therebetween). In one non-limiting formulation, the metal alloy includes 45-55 wt. % cobalt, and 45-55 wt. % molybdenum.

In accordance with another and/or alternative non-limiting aspect of the present disclosure, the metal alloy includes 90-99.5 wt. % niobium (and all values and ranges therebetween), and 0.5-10 wt. % zirconium (and all values and ranges therebetween). In one non-limiting formulation, the metal alloy includes 95-99.25 wt. % niobium, and 0.75-4 wt. % niobium.

In accordance with another and/or alternative non-limiting aspect of the present disclosure, the metal alloy includes 55-75 wt. % niobium (and all values and ranges therebetween), 18-40 wt. % tantalum (and all values and ranges therebetween), 1-7 wt. % tungsten (and all values and ranges therebetween), and 0.5-4 wt. % zirconium (and all values and ranges therebetween). In one non-limiting formulation, the metal alloy includes 60-70 wt. % niobium, 24-32 wt. % tantalum, 2-5 wt. % tungsten, and 0.75-3 wt. % zirconium.

In accordance with another and/or alternative non-limiting aspect of the present disclosure, the metal alloy includes less than about 5 wt. % (e.g., 0-4.999999 wt. % and all values and ranges therebetween) other metals and/or impurities. A high purity level of the metal alloy results in the formation of a more homogeneous alloy, which in turn results in a more uniform density throughout the metal alloy, and also results in the desired yield and ultimate tensile strengths of the metal alloy. In one non-limiting embodiment, the metal alloy includes less than about 0.5 wt. % other metals and/or impurities. In another non-limiting embodiment, the metal alloy includes less than about 0.2 wt. % other metals and/or impurities. In another non-limiting embodiment, the metal alloy includes less than about 0.1 wt. % other metals and/or impurities. In another non-limiting embodiment, the metal alloy includes less than about 0.05 wt. % other metals and/or impurities. In another non-limiting embodiment, the metal alloy includes less than about 0.01 wt. % other metals and/or impurities.

In accordance with another and/or alternative non-limiting aspect of the present disclosure, the medical device is generally designed to include at least about 5 wt. % of the metal alloy (e.g., 5-100 wt. % and all values and ranges therebetween). In one non-limiting embodiment of the disclosure, the medical device includes at least about 50 wt. % of the metal alloy. In another non-limiting embodiment of the disclosure, the medical device includes at least about 95 wt. % of the metal alloy. In one specific configuration, when the medical device includes an expandable frame, the expandable frame is formed of 50-100 wt. % (and all values and ranges therebetween) of the metal alloy, and typically 75-100 wt. % of the metal alloy.

In accordance with another and/or alternative non-limiting aspect of the present disclosure, the metal alloy can optionally be nitrided; however, this is not required. The nitrided layer on the metal alloy can function as a lubricating surface during the optional drawing of the metal alloy when partially or fully forming the medical device. After the metal alloy is nitrided, the metal alloy is typically cleaned; however, this is not required. During the nitriding process, the surface of the metal alloy is modified by the presence of nitrogen. The nitriding process can be by gas nitriding, salt bath nitriding, or plasma nitriding. In gas nitriding, the nitrogen diffuses onto the surface of the metal alloy, thereby creating a nitrided layer. The thickness and phase constitution of the resulting nitriding layers can be selected and the process optimized for the particular properties required. During gas nitriding, the metal alloy is generally nitrided in the presence of nitrogen gas or a nitrogen gas mixture (e.g., 90-99% vol. % N and 1-10 vol. % H, etc.) for at least 10 seconds at a temperature of at least about 400° C. (e.g., 400-1000° C. and all values and ranges therebetween). In one non-limiting nitriding process, the metal alloy is heated in the presence of nitrogen or a nitrogen-hydrogen mixture to a temperature of at least 400° C., and generally about 400-800° C. (and all values and ranges therebetween) for at least 10 seconds (e.g., 10 seconds to 60 minutes and all values and ranges therebetween), and generally about 1-30 minutes. In salt bath nitriding, a nitrogen-containing salt such as cyanide salt is used. During the salt bath nitriding, the metal alloy is generally exposed to temperatures of about 520-590° C. In plasma nitriding, the gas used for plasma nitriding is usually pure nitrogen. Plasma nitriding is often coupled with physical vapor deposition (PVD) process; however, this is not required. Plasma nitriding of the metal alloy generally occurs at a temperature of 220-630° C. (and all values and ranges therebetween). The metal alloy can optionally be exposed to argon and/or hydrogen gas prior to the nitriding process to clean and/or preheat the metal alloy. These gases can be optionally used to clean oxide layers and/or solvents from the surface of the metal alloy. During the nitriding process, the metal alloy can optionally be exposed to hydrogen gas to inhibit or prevent the formation of oxides on the surface of the metal alloy. The thickness of the nitrided surface layer is less than about 1 mm. In one non-limiting embodiment, the thickness of the nitride surface layer is at least about 50 nanometer and less than about 1 mm (and all values and ranges therebetween). In another non-limiting embodiment, the thickness of the nitrided surface layer is at least about 50 nanometer and less than about 0.1 mm. Generally, the weight percent of nitrogen in the nitrided surface layer is 0.0001-5 wt. % nitrogen (and all values and ranges therebetween). In one non-limiting embodiment, the weight percent of nitrogen in the nitrided surface layer is generally less than one of the primary components of the metal alloy, and typically less than each of the two primary components of the metal alloy. For example, when a metal alloy is nitrided, the weight percent of the nitrogen in the nitrided surface layer is less than a weight percent of the rhenium in the nitrided surface layer. In one non-limiting composition of the nitrided surface layer on a metal alloy (e.g., 47-55 wt. % rhenium, 10-46 wt. % molybdenum, 0.1-30 wt. % additional metal alloying agent), the nitrided surface layer comprises at least 40 wt. % rhenium, at least 8 wt. % molybdenum, and 0.0001-5 wt. % nitrogen (and all values and ranges therebetween). In one non-limiting embodiment of the disclosure, the surface of the metal alloy is nitrided prior to at least one drawing step for the metal alloy. In another non-limiting aspect of this disclosure, after the metal alloy has been annealed, the metal alloy is nitrided prior to being drawn. In another and/or alternative non-limiting embodiment, the metal alloy is cleaned to remove nitride compounds on the surface of the metal alloy prior to annealing the metal alloy. The nitride compounds can be removed by a variety of steps such as, but not limited to, grit blasting, polishing, etc. After the metal alloy has been annealed, the metal alloy can be again nitrided prior to one or more drawing steps; however, this is not required. As can be appreciated, the complete outer surface of the metal alloy can be nitrided or a portion of the outer surface of the metal alloy can be nitrided. Nitriding only selected portions of the outer surface of the metal alloy can be used to obtain different surface characteristics of the metal alloy; however, this is not required. As can be appreciated, the final formed metal alloy can include a nitrided outer surface. The nitriding process for the metal alloy can be used to increase surface hardness and/or wear resistance of the medical device, and/or to inhibit or prevent discoloration of the metal alloy (e.g., discoloration by oxidation, etc.). For example, the nitriding process can be used to increase the wear resistance of articulation surface or surfaces wear on the metal alloy used in the medical device to extend the life of the medical device, and/or increase the wear life of mating surfaces on the medical device (e.g., polyethylene liners of joint implants like knees, hips, shoulders, etc.), and/or to reduce particulate generation from use of the medical device, and/or to maintain the outer surface appearance of the metal alloy on the medical device.

In accordance with another and/or alternative non-limiting aspect of the present disclosure, the metal alloy, just prior to or after being partially or fully formed into the desired medical device, can optionally be cleaned, polished, sterilized, nitrided, etc., for final processing of the metal alloy. In one non-limiting embodiment of the disclosure, the metal alloy is electropolished. In one non-limiting aspect of this embodiment, the metal alloy is cleaned prior to being exposed to the polishing solution; however, this is not required. The cleaning process (when used) can be accomplished by a variety of techniques such as, but not limited to, 1) using a solvent (e.g., acetone, methyl alcohol, etc.) and wiping the metal alloy with a Kimwipe or other appropriate towel, and/or 2) by at least partially dipping or immersing the metal alloy in a solvent and then ultrasonically cleaning the metal alloy. As can be appreciated, the metal alloy can be cleaned in other or additional ways. In another and/or alternative non-limiting aspect of this embodiment, the polishing solution can include one or more acids. In yet another and/or alternative non-limiting aspect of this embodiment, the metal alloy is rinsed with water and/or a solvent and allowed to dry to remove polishing solution on the metal alloy.

In accordance with another and/or alternative non-limiting aspect of the present disclosure, the medical device can optionally contain and/or be coated with one or more agents that facilitate in the success of the medical device and/or treated area. The term “agent” includes, but is not limited to, a substance, pharmaceutical, biologic, veterinary product, drug, and analogs or derivatives otherwise formulated and/or designed to prevent, inhibit, and/or treat one or more clinical and/or biological events, and/or to promote healing. Non-limiting examples of clinical events that can be addressed by one or more agents include, but are not limited to, viral, fungus and/or bacterial infection; vascular diseases and/or disorders, digestive diseases and/or disorders, reproductive diseases and/or disorders, lymphatic diseases and/or disorders, cancer, implant rejection, pain, nausea, swelling, arthritis, bone diseases and/or disorders, organ failure, immunity diseases and/or disorders, cholesterol problems, blood diseases and/or disorders, lung diseases and/or disorders, heart diseases and/or disorders, brain diseases and/or disorders, neuralgia diseases and/or disorders, kidney diseases and/or disorders, ulcers, liver diseases and/or disorders, intestinal diseases and/or disorders, gallbladder diseases and/or disorders, pancreatic diseases and/or disorders, psychological disorders, respiratory diseases and/or disorders, gland diseases and/or disorders, skin diseases and/or disorders, hearing diseases and/or disorders, oral diseases and/or disorders, nasal diseases and/or disorders, eye diseases and/or disorders, fatigue, genetic diseases and/or disorders, burns, scarring and/or scars, trauma, weight diseases and/or disorders, addiction diseases and/or disorders, hair loss, cramp, muscle spasms, tissue repair, nerve repair, neural regeneration, and/or the like. The type and/or amount of agent included in the medical device and/or coated on medical device can vary. When two or more agents are included in and/or coated on the medical device, the amount of the two or more agents can be the same or different. The one or more agents can be coated on and/or impregnated in the medical device by a variety of mechanisms such as, but not limited to, spraying (e.g., atomizing spray techniques, etc.), flame spray coating, powder deposition, dip coating, flow coating, dip-spin coating, roll coating (direct and reverse), sonication, brushing, plasma deposition, depositing by vapor deposition, MEMS technology, and rotating mold deposition. In another and/or alternative non-limiting embodiment of the disclosure, the type and/or amount of agent included on, in, and/or in conjunction with the medical device is generally selected for the treatment of one or more medical treatments. The amount of two or more agents on, in, and/or used in conjunction with the medical device can be the same or different. The one or more agents, when used on and/or in the medical device, can optionally be released in a controlled manner so the area in question to be treated is provided with the desired dosage of agent over a sustained period of time. As can be appreciated, controlled release of one or more agents on the medical device is not always required and/or desirable. As such, one or more of the agents on and/or in the medical device can be uncontrollably released from the medical device during and/or after insertion of the medical device in the treatment area. It can also be appreciated that one or more agents on and/or in the medical device can be controllably released from the medical device and one or more agents on and/or in the medical device can be uncontrollably released from the medical device. It can also be appreciated that one or more agents on and/or in one region of the medical device can be controllably released from the medical device and one or more agents on and/or in the medical device can be uncontrollably released from another region on the medical device. As such, the medical device can be designed such that 1) all the agent on and/or in the medical device is controllably released, 2) some of the agent on and/or in the medical device is controllably released and some of the agent on the medical device is non-controllably released, or 3) none of the agent on and/or in the medical device is controllably released. The medical device can also be designed such that the rate of release of the one or more agents from the medical device is the same or different. The medical device can also be designed such that the rate of release of the one or more agents from one or more regions on the medical device is the same or different. Non-limiting arrangements that can be used to control the release of one or more agents from the medical device include 1) at least partially coating one or more agents with one or more polymers, 2) at least partially incorporating and/or at least partially encapsulating one or more agents into and/or with one or more polymers, and/or 3) inserting one or more agents in pores, passageway, cavities, etc., in the medical device and at least partially coat or cover such pores, passageway, cavities, etc., with one or more polymers. As can be appreciated, other or additional arrangements can be used to control the release of one or more agents from the medical device. The one or more polymers, when used to at least partially control the release of one or more agents from the medical device, can be porous or non-porous. The one or more agents can be inserted into and/or applied to one or more surface structures and/or micro-structures on the medical device, and/or be used to at least partially form one or more surface structures and/or micro-structures on the medical device. As such, the one or more agents on the medical device can be 1) coated on one or more surface regions of the medical device, 2) inserted and/or impregnated in one or more surface structures and/or micro-structures, etc., of the medical device, and/or 3) form at least a portion or be included in at least a portion of the structure of the medical device. When the one or more agents are coated on the medical device, the one or more agents can 1) be directly coated on one or more surfaces of the medical device, 2) be mixed with one or more coating polymers or other coating materials and then at least partially coated on one or more surfaces of the medical device, 3) be at least partially coated on the surface of another coating material that has been at least partially coated on the medical device, and/or 4) be at least partially encapsulated between a) a surface or region of the medical device and one or more other coating materials and/or b) two or more other coating materials. As can be appreciated, many other coating arrangements can be additionally or alternatively used. When the one or more agents are optionally inserted and/or impregnated in one or more internal structures, surface structures and/or micro-structures of the medical device, 1) one or more other coating materials can be applied at least partially over the one or more internal structures, surface structures, and/or micro-structures of the medical device, and/or 2) one or more polymers can be combined with one or more agents. As such, the one or more agents can be 1) embedded in the structure of the medical device, 2) positioned in one or more internal structures of the medical device, 3) encapsulated between two polymer coatings, 4) encapsulated between the base structure and a polymer coating, 5) mixed in the base structure of the medical device that includes at least one polymer coating, or 6) one or more combinations of 1, 2, 3, 4, and/or 5. In addition or alternatively, the one or more coating of the one or more polymers on the medical device can include 1) one or more coatings of non-porous polymers, 2) one or more coatings of a combination of one or more porous polymers and one or more non-porous polymers, 3) one or more coating of porous polymer, or 4) one or more combinations of options 1, 2, and 3. As can be appreciated, different agents can optionally be located in and/or between different polymer coating layers and/or on the structure of the medical device. As can also be appreciated, many other and/or additional coating combinations and/or configurations can be used. The concentration of one or more agents, the type of polymer, the type and/or shape of internal structures in the medical device, and/or the coating thickness of one or more agents can be used to control the release time, the release rate, and/or the dosage amount of one or more agents; however, other or additional combinations can be used. As such, the agent and polymer system combination and location on the medical device can be numerous. As can also be appreciated, one or more agents can be deposited on the top surface of the medical device to provide an initial uncontrolled burst effect of the one or more agents prior to the 1) controlled release of the one or more agents through one or more layers of a polymer system that include one or more non-porous polymers, and/or 2) uncontrolled release of the one or more agents through one or more layers of a polymer system. The one or more agents and/or polymers can be coated on the medical device by a variety of mechanisms such as, but not limited to, spraying (e.g., atomizing spray techniques, etc.), dip coating, roll coating, sonication, brushing, plasma deposition, and/or depositing by vapor deposition.

In accordance with another and/or alternative non-limiting aspect of the present disclosure, a variety of polymers can optionally be coated on the medical device and/or be used to form at least a portion of the medical device. The one or more polymers can be used on the medical device for a variety of reasons such as, but not limited to, 1) forming a portion of the medical device, 2) improving a physical property of the medical device (e.g., improve strength, improve durability, improve biocompatibility, reduce friction, etc.), 3) forming a protective coating on one or more surface structures on the medical device, 4) at least partially forming one or more surface structures on the medical device, and/or 5) at least partially controlling a release rate of one or more agents from the medical device. As can be appreciated, the one or more polymers can have other or additional uses on the medical device. The one or more polymers can be porous, non-porous, biostable, biodegradable (i.e., dissolves, degrades, is absorbed, or any combination thereof in the body), and/or biocompatible. When the medical device is coated with one or more polymers, the polymer can include 1) one or more coatings of non-porous polymers, 2) one or more coatings of a combination of one or more porous polymers and one or more non-porous polymers, 3) one or more coatings of one or more porous polymers and one or more coatings of one or more non-porous polymers, 4) one or more coating of porous polymer, or 5) one or more combinations of options 1, 2, 3, and 4. The thickness of one or more of the polymer layers can be the same or different. When one or more layers of polymer are coated onto at least a portion of the medical device, the one or more coatings can be applied by a variety of techniques such as, but not limited to, vapor deposition and/or plasma deposition, spraying, dip-coating, roll coating, sonication, atomization, brushing, and/or the like; however, other or additional coating techniques can be used. The one or more polymers that can be coated on the medical device and/or used to at least partially form the medical device can be polymers that are considered to be biodegradable, bioresorbable, or bioerodable; polymers that are considered to be biostable; and/or polymers that can be made to be biodegradable and/or bioresorbable with modification. The thickness of each polymer layer is generally at least about 0.01 μm and is generally less than about 150 μm (e.g., 0.01 μm to 150 μm and all values and ranges therebetween); however, other thicknesses can be used. In one non-limiting embodiment, the thickness of a polymer layer and/or layer of agent is about 0.02-75 μm, more particularly about 0.05-50 μm, and even more particularly about 1-30 μm. As can be appreciated, other thicknesses can be used.

In accordance with another and/or alternative non-limiting aspect of the present disclosure, the medical device, when including and/or is coated with one or more agents, can include and/or can be coated with one or more agents that are the same or different in different regions of the medical device and/or have differing amounts and/or concentrations in differing regions of the medical device. For instance, the medical device can 1) be coated with and/or include one or more biologicals on at least one portion of the medical device and at least another portion of the medical device is not coated with and/or includes agent; 2) be coated with and/or include one or more biologicals on at least one portion of the medical device that is different from one or more biologicals on at least another portion of the medical device; and/or 3) be coated with and/or include one or more biologicals at a concentration on at least one portion of the medical device that is different from the concentration of one or more biologicals on at least another portion of the medical device.

dance with another and/or alternative non-limiting aspect of the present disclosure, one or more portions of the medical device can optionally 1) include the same or different agents, 2) include the same or different amount of one or more agents, 3) include the same or different polymer coatings, 4) include the same or different coating thicknesses of one or more polymer coatings, 5) have one or more portions of the medical device controllably release and/or uncontrollably release one or more agents, and/or 6) have one or more portions of the medical device controllably release one or more agents and one or more portions of the medical device uncontrollably release one or more agents.

In accordance with another and/or alternative non-limiting aspect of the present disclosure, the medical device can optionally include a marker material that facilitates enabling the medical device to be properly positioned in a body passageway. The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.). The marker material can form all or a portion of the medical device and/or be coated on one or more portions (flaring portion and/or body portion, at ends of medical device, at or near transition of body portion and flaring section, etc.) of the medical device. The location of the marker material can be on one or multiple locations on the medical device. The size of the one or more regions including the marker material can be the same or different. The marker material can be spaced at defined distances from one another to form ruler-like markings on the medical device to facilitate in the positioning of the medical device in a body passageway. The marker material can be a rigid or flexible material. The marker material can be a biostable or biodegradable material.

In accordance with another and/or alternative non-limiting aspect of the present disclosure, the medical device or one or more regions of the medical device can optionally be constructed by use of one or more microelectromechanical manufacturing (MEMS) techniques (e.g., micro-machining, laser micro-machining, micro-molding, etc.); however, other or additional manufacturing techniques can be used.

In accordance with another and/or alternative non-limiting aspect of the present disclosure, the medical device can optionally include one or more surface structures (e.g., pore, channel, pit, rib, slot, notch, bump, teeth, needle, well, hole, groove, etc.). These structures can be at least partially formed by MEMS (e.g., micro-machining, etc.) technology and/or other types of technology.

In accordance with another and/or alternative non-limiting aspect of the present disclosure, the medical device can optionally include one or more micro-structures (e.g., micro-needle, micro-pore, micro-cylinder, micro-cone, micro-pyramid, micro-tube, micro-parallelopiped, micro-prism, micro-hemisphere, teeth, rib, ridge, ratchet, hinge, zipper, zip-tie like structure, etc.) on the surface of the medical device. As defined herein, a “micro-structure” is a structure having at least one dimension (e.g., average width, average diameter, average height, average length, average depth, etc.) that is no more than about 2 mm, and typically no more than about 1 mm. As can be appreciated, when the medical device includes one or more surface structures, 1) all the surface structures can be micro-structures, 2) all the surface structures can be non-micro-structures, or 3) a portion of the surface structures can be micro-structures and a portion can be non-micro-structures. Typically, the micro-structures (when formed) extend from or into the outer surface no more than about 400 microns (0.01-400 microns and all values and ranges therebetween), and more typically less than about 300 microns, and more typically about 15-250 microns; however, other sizes can be used. The micro-structures can be clustered together or disbursed throughout the surface of the medical device. Similar shaped and/or sized micro-structures and/or surface structures can be used, or different shaped and/or sized micro-structures can be used. When one or more surface structures and/or micro-structures are designed to extend from the surface of the medical device, the one or more surface structures and/or micro-structures can be formed in the extended position and/or be designed to extend from the medical device during and/or after deployment of the medical device in a treatment area. The micro-structures and/or surface structures can be designed to contain and/or be fluidly connected to a passageway, cavity, etc.; however, this is not required. The one or more surface structures and/or micro-structures can be used to engage and/or penetrate surrounding tissue or organs once the medical device has been positioned on and/or in a patient; however, this is not required. The one or more surface structures and/or micro-structures can be used to facilitate in forming and maintaining a shape of a medical device. In one non-limiting embodiment, the one or more surface structures and/or micro-structures can be at least partially formed of an agent and/or be formed of a polymer. One or more of the surface structures and/or micro-structures can include one or more internal passageways that can include one or more materials (e.g., agent, polymer, etc.); however, this is not required. The one or more coatings and/or one or more surface structures and/or micro-structures of the medical device can be used for a variety of purposes such as, but not limited to, 1) increasing the bonding and/or adhesion of one or more agents, adhesives, marker materials, and/or polymers to the medical device, 2) changing the appearance or surface characteristics of the medical device, and/or 3) controlling the release rate of one or more agents. The one or more micro-structures and/or surface structures can be biostable, biodegradable, etc. The medical device or one or more regions of the medical device can be at least partially covered and/or filled with a protective material to at least partially protect one or more regions of the medical device, and/or one or more micro-structures, and/or surface structures on the medical device from damage. The protective material can include one or more polymers previously identified above. The protective material can be 1) biostable and/or biodegradable and/or 2) porous and/or non-porous.

In accordance with another and/or alternative non-limiting aspect of the present disclosure, the medical device can optionally be an expandable device that can be expanded by use of some other device (e.g., balloon, etc.). The expandable medical device can be fabricated from a material that has no or substantially no shape-memory characteristics.

In accordance with another and/or alternative non-limiting aspect of the present disclosure, there is optionally provided a near net process for a frame or other metal component of the medical device. In one non-limiting embodiment of the disclosure, there is provided a method of powder pressing materials and increasing the strength post-sintering by imparting additional cold work. In one non-limiting embodiment, the green part is pressed and then sintered. Thereafter, the sintered part is again pressed to increase its mechanical strength by imparting cold work into the pressed and sintered part. Generally, the temperature during the pressing process after the sintering process is 20-100° C. (and all values and ranges therebetween), typically 20-80° C., and more typically 20-40° C. As defined herein, cold working occurs at a temperature of no more than 150° C. (e.g., 10-150° C. and all values and ranges therebetween). The change in the shape of the repressed post-sintered part needs to be determined so the final part (pressed, sintered and re-pressed) meets the dimensional requirements of the final formed part. For a metal alloy, a prepress pressure of 1-300 tsi (1 ton per square inch) (and all values and ranges therebetween) can be used followed by a sintering process of at least 1600° C. (e.g., 1600-2600° C. and all values and ranges therebetween) and a post sintering press at a pressure of 1-300 tsi (and all values and ranges therebetween) at a temperature of at least 20° C. (e.g., 20-100° C. and all values and ranges therebetween; 20-40° C., etc.). There is also provided a process of increasing the mechanical strength of a pressed metal part by repressing the post-sintered part to add additional cold work into the material, thereby increasing its mechanical strength. There is also provided a process of powder pressing to a near net or final part using metal powder. In one non-limiting embodiment, the metal powder used to form the near net or final part includes a one or more elements of rhenium, molybdenum, titanium, cobalt, iron, boron, nickel, tungsten, tantalum, aluminum, vanadium, chromium, niobium, silicon, manganese, carbon, zirconium, iridium, titanium, bismuth, and yttrium.

In accordance with another and/or alternative non-limiting aspect of the present disclosure, there is optionally provided a press of near net or finished part composite. The process of pressing metals into near net of finished parts is well established; however, pressing a composite structure formed of metal powder and polymer for purposes of making complex part geometries and foam-like structures is new. Similarly, using a pressing process to impart particular biologic substances into the metal matrix is also new. In one non-limiting embodiment, there is provided a process of creating a metal part with pre-defined voids to create a trabecular or foam structure composed of mixing a metal and polymer powder, pressing the powder into a finished part or semi-finished green part, and then sintering the part under which conditions the polymer leaves the metal behind through a process of thermal degradation of the polymer. The resulting part has a porosity associated with the size of the polymer particles as well as the homogeneity of the mixture upon pressing prior to sintering. In another non-limiting embodiment, there is provided a process by which a residual of the polymer is left behind after thermal degradation, on the metal substrate, and the polymer residual has some desired biological affect (e.g., masking the metal from the body by encapsulation, promotion of cellular attachment and growth). The polymer and metal powders can be of varying sizes to create multiple voids—some large to create a pathway for cellular growth, and some small to create a ruff surface to promote cellular attachment. As can be appreciated, the polymer can be uniformly or non-uniformly dispersed with the metal powder. For example, if the final formed part is to have a uniform density and pore structure, the polymer material is uniformly dispersed with the metal powder prior to consolidating and pressing the polymer and metal powders together and then subsequently sintering together the metal powder to form the metal part or medical device. Alternatively, if the formed metal part or medical device is to have one or more channels, passageways, and/or voids on the outer surface and/or within the formed part or medical device, at least a portion of the polymer is not uniformly distributed with the metal powder, but instead is concentrated or forms all of the region that is to be the one or more channels, passageways, and/or voids on the outer surface and/or within the formed part or medical device such that when the polymer and metal powder is sintered, some or all of the polymer is degraded and removed from the part or medical device, thereby forming such one or more channels, passageways, and/or voids on the outer surface and/or within the formed part or medical device. As such, the use of the polymer in combination with metal powder and subsequent pressing and sintering can be used to form novel and customized shapes for the medical device or the near net form of the medical device. Generally, the polymer constitutes about 0.1-70 vol. % (and all values and ranges therebetween) of the consolidated and pressed material prior to the sintering step, typically the polymer constitutes about 1-60 vol. % of the consolidated and pressed material prior to the sintering step, more typically the polymer constitutes about 2-50 vol. % of the consolidated and pressed material prior to the sintering step, and even more typically the polymer constitutes about 2-45 vol. % of the consolidated and pressed material prior to the sintering step. As such, if the polymer constitutes about 5 vol. % of the consolidated and pressed material prior to the sintering step, if after the sintering step at least 99% of the polymer is degraded and removed from the part or medical device, then the part could include up to about 5 vol. % cavities and/or passageways in the part or medical device. The type of polymer and the type of metal powder is non-limiting. The polymer and metal powders can be of varying sizes to create multiple voids/passageways/channels which can be used to create a pathway for cellular growth, create a ruff surface to promote cellular attachment, have a biological agent inserted into one or more of the voids/passageways/channels, have biological material inserted into one or more of the voids/passageways/channels, etc. In one non-limiting embodiment, the average particle size of the polymer is greater than the average particle size of the metal powder.

In accordance with another and/or alternative non-limiting aspect of the present disclosure, after the sintering process, at least 98 vol. % of the polymer is thermally degraded and/or removed from the sintered material, typically at least 99 vol. % of the polymer is thermally degraded and/or removed from the sintered material, more typically at least 99.5 vol. % of the polymer is thermally degraded and/or removed from the sintered material, still even more typically at least 99.9 vol. % of the polymer is thermally degraded and/or removed from the sintered material, and even still more typically at least 99.95 vol. % of the polymer is thermally degraded and/or removed from the sintered material. The resulting part or medical device has a porosity associated with the size of the polymer particles as well as the homogeneity of the mixture upon pressing prior to sintering.

In accordance with another and/or alternative non-limiting aspect of the present disclosure, after the sintering process, some of the polymer remains in the sintered part of the medical device. The remaining polymer in the sintered part of the medical device can optionally have some desired biological affect (e.g., masking the metal from the body by encapsulation, promotion of cellular attachment and growth). The remaining polymer can optionally include one or more biological agents that remain active after the sintering process. In one non-limiting embodiment, after the sintering process, about 5-97.5 vol. % (and all values and ranges therebetween) of the polymer is thermally degraded and/or removed from the sintered material, typically about 10-95 vol. % of the polymer is thermally degraded and removed from the sintered material, and more typically about 10-80 vol. % of the polymer is thermally degraded and removed from the sintered material.

In accordance with another and/or alternative non-limiting aspect of the present disclosure, the metal alloy used to at least partially form the medical device is initially formed into a blank, a rod, a tube, etc., and then finished into final form by one or more finishing processes. The metal alloy blank, rod, tube, etc., can be formed by various techniques such as, but not limited to, 1) melting the metal alloy and/or metals that form the metal alloy (e.g., vacuum arc melting, etc.) and then extruding and/or casting the metal alloy into a blank, rod, tube, etc., 2) melting the metal alloy and/or metals that form the metal alloy, forming a metal strip, and then rolling and welding the strip into a blank, rod, tube, etc., or 3) consolidating the metal powder of the metal alloy and/or metal powder of metals that form the metal alloy into a blank, rod, tube, etc. When the metal alloy is formed into a blank, the shape and size of the blank is non-limiting. In one non-limiting process, the near net medical device, blank, rod, tube, etc., can be formed from one or more ingots of metal or metal alloy. In one non-limiting process, an arc melting process (e.g., vacuum arc melting process, etc.) can be used to form the near net medical device, blank, rod, tube, etc. In another non-limiting process, rhenium powder and tungsten powder and optionally molybdenum powder can be placed in a crucible (e.g., silica crucible, etc.) and heated under a controlled atmosphere (e.g., vacuum environment, carbon monoxide environment, hydrogen and argon environment, helium, argon, etc.) by an induction melting furnace to form the near net medical device, blank, rod, tube, etc. It can be appreciated that other or additional processes can be used to form the metal alloy. In one non-limiting embodiment, the average particle size of the metal powders is less than about 230 mesh (e.g., less than 63 microns). In another and/or alternative non-limiting embodiment, the average particle size of the metal powders is about 2-63 microns, and more particularly about 5-40 microns. As can be appreciated, smaller average particle sizes can be used. The purity of the metal powders should be selected so that the metal powders contain very low levels of carbon, oxygen, and nitrogen. Typically, the carbon content of the metal powder used to form the metal alloy is less than about 100 ppm, the oxygen content is less than about 50 ppm, and the nitrogen content is less than about 20 ppm. Typically, metal powder used to form the metal alloy has a purity grade of at least 99.9 and more typically at least about 99.95. The blend of metal powder is then pressed together to form a solid solution of the metal alloy into a near net medical device, blank, rod, tube, etc. Typically, the pressing process is by an isostatic process (i.e., uniform pressure applied from all sides on the metal powder); however other processes can be used. When the metal powders are pressed together isostatically, cold isostatic pressing (CIP) is typically used to consolidate the metal powders; however, this is not required. The pressing process can be performed in an inert atmosphere, an oxygen-reducing atmosphere (e.g., hydrogen, argon and hydrogen mixture, etc.), and/or under a vacuum; however, this is not required. The average density of the near net medical device, blank, rod, tube, etc., that is achieved by pressing together the metal powders is about 80-95% (and all values and ranges therebetween) of the final average density of the near net medical device, blank, rod, tube, etc., or about 70-96% (and all values and ranges therebetween) the minimum theoretical density of the metal alloy. Pressing pressures of at least about 300 MPa are generally used. Generally, the pressing pressure is about 400-700 MPa; however, other pressures can be used. After the metal powders are pressed together, the pressed metal powders are sintered at a temperature of at least 1600° C. (e.g., 1600-3500° C. and all values and ranges therebetween) to partially or fully fuse the metal powders together to form the near net medical device, blank, rod, tube, etc. The sintering of the consolidated metal powder can be performed in an oxygen-reducing atmosphere (e.g., helium, argon, hydrogen, argon and hydrogen mixture, etc.), and/or under a vacuum; however, this is not required. At the high sintering temperatures, a high hydrogen atmosphere will reduce both the amount of carbon and oxygen in the formed near net medical device, blank, rod, tube, etc. The sintered metal powder generally has an as-sintered average density of about 90-99% the minimum theoretical density of the metal alloy. Typically, the sintered metal alloy has a final average density of at least about 5 μm/cc, and typically at least about 8.3 μm/cc, and can be up to or greater than about 16 μm/cc; however, this is not required. The density of the formed near net medical device, blank, rod, tube, etc., will generally depend on the type of metal alloy used.

In accordance with another and/or alternative non-limiting aspect of the present disclosure, the near net medical device, blank, rod, tube, etc., can optionally be cleaned and/or polished after the near net medical device, blank, rod, tube, etc., has been form; however, this is not required. Typically, the near net medical device, blank, rod, tube, etc., is cleaned and/or polished prior to being further processed; however, this is not required. When the near net medical device, blank, rod, tube, etc., is resized and/or annealed, the resized and/or annealed, the near net medical device, blank, rod, tube, etc., is typically cleaned and/or polished prior to and/or after each or after a series of resizing and/or annealing processes; however, this is not required. The cleaning and/or polishing of the near net medical device, blank, rod, tube, etc., is used to remove impurities and/or contaminants from the surfaces of the near net medical device, blank, rod, tube, etc. Impurities and contaminants can become incorporated into the metal alloy during the processing of the near net medical device, blank, rod, tube, etc. The inadvertent incorporation of impurities and contaminants in the near net medical device, blank, rod, tube, etc., can result in an undesired amount of carbon, nitrogen and/or oxygen, and/or other impurities in the metal alloy. The inclusion of impurities and contaminants in the metal alloy can result in premature micro-cracking of the metal alloy and/or an adverse effect on one or more physical properties of the metal alloy (e.g., decrease in tensile elongation, increased ductility, increased brittleness, etc.). The cleaning of the metal alloy can be accomplished by a variety of techniques such as, but not limited to, 1) using a solvent (e.g., acetone, methyl alcohol, etc.) and wiping the metal alloy with a Kimwipe or other appropriate towel, 2) by at least partially dipping or immersing the metal alloy in a solvent and then ultrasonically cleaning the metal alloy, and/or 3) by at least partially dipping or immersing the metal alloy in a pickling solution. As can be appreciated, the metal alloy can be cleaned in other or additional ways. If the metal alloy is to be polished, the metal alloy is generally polished by use of a polishing solution that typically includes an acid solution; however, this is not required.

In accordance with another and/or alternative non-limiting aspect of the present disclosure, the near net medical device, blank, rod, tube, etc., can be resized to the desired dimension of the medical device. In one non-limiting embodiment, the cross-sectional area or diameter of the near net medical device, blank, rod, tube, etc., is reduced to a final near net medical device, blank, rod, tube, etc. dimension in a single step or by a series of steps. The reduction of the outer cross-sectional area or diameter of the near net medical device, blank, rod, tube, etc., may be obtained by centerless grinding, turning, electropolishing, drawing process, grinding, laser cutting, shaving, polishing, EDM cutting, etc. The outer cross-sectional area or diameter size of the near net medical device, blank, rod, tube, etc., can be reduced by the use of one or more drawing processes; however, this is not required. During the drawing process, care should be taken to not form micro-cracks in the near net medical device, blank, rod, tube, etc., during the reduction of the near net medical device, blank, rod, tube, etc., outer cross-sectional area or diameter.

One non-limiting object of the present disclosure is the provision of metal alloy in accordance with the present disclosure that can be used to partially or fully form a medical device.

Another and/or alternative non-limiting object of the present disclosure is the provision of a medical device that is partially or fully formed of the metal alloy of the present disclosure which medical device has improved procedural success rates.

Another and/or alternative non-limiting object of the present disclosure is the provision of a method and process for forming the metal alloy in accordance with the present disclosure that inhibits or prevents the formation of micro-cracks during the processing of the metal alloy.

Another and/or alternative non-limiting object of the present disclosure is the provision of a medical device that is partially or fully formed of the metal alloy in accordance with the present disclosure wherein the medical device has improved physical properties.

Another and/or alternative non-limiting object of the present disclosure is the provision of a medical device that is at least partially formed of the metal alloy in accordance with the present disclosure wherein the medical device has increased strength and/or hardness.

Another and/or alternative non-limiting object of the present disclosure is the provision of a medical device that at least partially includes the metal alloy in accordance with the present disclosure which metal alloy enables the medical device to be formed with less material without sacrificing the strength of the medical device as compared to prior medical devices.

Another and/or alternative non-limiting object of the present disclosure is the provision of a method and process for forming the metal alloy in accordance with the present disclosure which inhibits or prevents the formation of micro-cracks during the processing of the metal alloy into a medical device.

Another and/or alternative non-limiting object of the present disclosure is the provision of a method and process for forming the metal alloy in accordance with the present disclosure that inhibits or prevents crack propagation and/or fatigue failure of the metal alloy.

Another and/or alternative non-limiting object of the present disclosure is the provision of a medical device that has had a nitriding process to form a nitrided layer on the outer surface of the metal alloy.

Another and/or alternative non-limiting object of the present disclosure is the provision of a medical device wherein the metal alloy has been subjected to a swaging process.

Another and/or alternative non-limiting object of the present disclosure is the provision of a medical device that includes a metal alloy wherein the metal alloy has been subjected to a cold-working process.

Another and/or alternative non-limiting object of the present disclosure is the provision of a medical device that includes a metal alloy that has increased strength and/or hardness compared with standard stainless steel, standard chromium-cobalt alloys, or standard titanium alloys.

Another and/or alternative non-limiting object of the present disclosure is the provision of a medical device that includes a metal alloy, thereby requiring a lesser quantity of metal alloy to achieve similar strengths compared to medical devices formed of different metals.

Another and/or alternative non-limiting object of the present disclosure is the provision of a medical device that includes a metal alloy wherein the medical device has a smaller crimped profile compared to medical devices formed of different metals.

Another and/or alternative non-limiting object of the present disclosure is the provision of a medical device that includes a metal alloy wherein the medical device has thinner walls and still achieves a similar or improved radial strength compared with thicker walled medical devices formed of standard stainless steel, standard chromium-cobalt alloy, or standard titanium alloys.

Another and/or alternative non-limiting object of the present disclosure is the provision of a medical device that includes a metal alloy wherein the medical device has improved stress-strain properties, bendability properties, elongation properties, and/or flexibility properties compared to medical devices formed of standard stainless steel, standard titanium steel, or standard chromium-cobalt alloys.

Another and/or alternative non-limiting object of the present disclosure is the provision of a medical device that includes a metal alloy wherein the medical device has an increased life compared to medical devices formed of standard stainless steel, standard titanium steel, or standard chromium-cobalt alloys.

Another and/or alternative non-limiting object of the present disclosure is the provision of a medical device that includes a metal alloy wherein the medical device has a reduced degree of recoil during the crimping and/or expansion of the medical device compared with standard stainless steel, standard chromium-cobalt alloys, or standard titanium alloys.

Another and/or alternative non-limiting object of the present disclosure is the provision of a medical device that includes a metal alloy wherein the medical device better conforms to an irregularly shaped body passageway when expanded in the body passageway as compared to a medical device formed by standard stainless steel, standard chromium-cobalt alloys, or standard titanium alloys.

Another and/or alternative non-limiting object of the present disclosure is the provision of a medical device that includes a metal alloy wherein the medical device has improved fatigue ductility when subjected to cold-working as compared to the cold-working of standard stainless steel, standard chromium-cobalt alloys, or standard titanium alloys.

Another and/or alternative non-limiting object of the present disclosure is the provision of a medical device that includes a metal alloy wherein the medical device has improved durability compared to standard stainless steel, standard chromium-cobalt alloys, or standard titanium alloys.

Another and/or alternative non-limiting object of the present disclosure is the provision of a medical device that includes a metal alloy wherein the medical device has improved hydrophilicity compared to standard stainless steel, standard chromium-cobalt alloys, or standard titanium alloys.

Another and/or alternative non-limiting object of the present disclosure is the provision of a medical device that includes a metal alloy wherein the medical device has reduced ion release in the body passageway compared to standard stainless steel, standard chromium-cobalt alloys, or standard titanium alloys.

Another and/or alternative non-limiting object of the present disclosure is the provision of a medical device that includes a metal alloy wherein the medical device is less of an irritant to the body than standard stainless steel or standard cobalt-chromium alloy or standard titanium alloys, thus can result in reduced inflammation, faster healing, and increased success rates of the medical device.

In accordance with another and/or alternative non-limiting aspect of the present disclosure, the medical device can be at least partially or fully formed from by 3D printing.

In accordance with another and/or alternative non-limiting aspect of the present disclosure, the unique combination of the metals in the metal alloy of the present disclosure with the desired purity and composition and the desired grain size results in 1) a medical device having the desired high ductility at about room temperature, 2) a medical device having the desired amount of tensile elongation, 3) a homogeneous or solid solution of a metal alloy having high radiopacity, 4) a reduction or prevention of micro-crack formation and/or breaking of the metal alloy of the present disclosure tube when the tube is sized and/or cut to form the medical device or portion of the medical device (e.g., frame of the medical device, etc.), 5) a reduction or prevention of micro-crack formation and/or breaking of the medical device or portion of the medical device (e.g., frame of the medical device, etc.) when the medical device or portion of the medical device (e.g., frame of the medical device, etc.) is crimped, 6) a reduction or prevention of micro-crack formation and/or breaking of the medical device or portion of the medical device (e.g., frame of the medical device, etc.) when the medical device is bent and/or expanded in a body passageway, 7) a medical device having the desired ultimate tensile strength and yield strength, 8) a medical device or portion of the medical device (e.g., frame of the medical device, etc.) having very thin wall thicknesses and still having the desired radial forces needed to retain the medical device or portion of the medical device (e.g., frame of the medical device, etc.) on an open state when expanded, 9) a medical device or portion of the medical device (e.g., frame of the medical device, etc.) exhibiting less recoil when the medical device or portion of the medical device (e.g., frame of the medical device, etc.) is crimped onto a delivery system and/or expanded in a body passageway, 10) a medical device exhibiting improved conformity to the shape of the treatment area in the body passageway when the medical device is expanded in a body passageway, 11) a medical device exhibiting improved fatigue ductility, 12) a medical device exhibiting reduced foreshortening when expanded, and/or 13) a medical device that exhibits improved durability.

These and other advantages will become apparent to those skilled in the art upon the reading and following of this description.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall therebetween.

To aid the Patent Office and any readers of this application and any resulting patent in interpreting the claims appended hereto, applicants do not intend any of the appended claims or claim elements to invoke 35 U.S.C. 112 (f) unless the words “means for” or “step for” are explicitly used in the particular claim.

1-42. (canceled)

43. A medical device that is partially or fully formed of a metal alloy; said medical device is a device for implantation in a body passageway, a spine, or into a bone; said metal alloy includes least 15 awt. % rhenium; said metal alloy further includes a primary metal additive selected from one or more of chromium, molybdenum, niobium, tantalum, and/or tungsten; a combined weight percent of rhenium and metal additive constitutes at least 80 wt. % of said metal alloy; said metal alloy includes up to 20 wt. % of a secondary metal additive selected from one or more of aluminum, bismuth, calcium, carbon, copper, gold, hafnium, iridium, iron, lanthanum, magnesium, manganese, nickel, osmium, platinum, rare earth metals, rhodium, ruthenium, silicon, silver, technetium, tin, titanium, vanadium, yttrium, zinc, and zirconium.

44. The medical device as defined in claim 43, wherein said primary metal additive is selected from two or more of chromium, molybdenum, niobium, tantalum, and/or tungsten.

45. The medical device as defined in claim 43, wherein said metal alloy includes chromium; said metal alloy includes less than 50 wt. % chromium.

46. The medical device as defined in claim 44, wherein said metal alloy includes chromium; said metal alloy includes less than 50 wt. % chromium.

47. The medical device as defined in claim 43, wherein said metal alloy includes molybdenum; said metal alloy includes less than 80 wt. % chromium; a combined weight of rhenium and molybdenum in said metal alloy is at least 50 wt. %.

48. The medical device as defined in claim 46, wherein said metal alloy includes molybdenum; said metal alloy includes less than 80 wt. % chromium; a combined weight of rhenium and molybdenum in said metal alloy is at least 50 wt. %.

49. The medical device as defined in claim 43, wherein said metal alloy includes zirconium, and molybdenum.

50. The medical device as defined in claim 43, wherein said metal alloy includes cobalt, and molybdenum.

51. The medical device as defined in claim 43, wherein said metal alloy includes niobium, and zirconium.

52. The medical device as defined in claim 43, wherein said metal alloy includes niobium, tantalum, and zirconium.

53. The medical device as defined in claim 43, wherein said medical device includes an expandable frame that is at least partially formed of said metal alloy.

54. A medical device in the formed of an orthopedic rod, stent or frame for a prosthetic valve; said medical device is partially or fully formed of a metal alloy; said metal alloy includes least 15 awt. % rhenium; said metal alloy further includes a primary metal additive selected from two or more of chromium, molybdenum, niobium, tantalum, and/or tungsten; a combined weight percent of rhenium and metal additive constitutes at least 80 wt. % of said metal alloy; said metal alloy includes up to 20 wt. % of a secondary metal additive selected from one or more of aluminum, bismuth, calcium, carbon, copper, gold, hafnium, iridium, iron, lanthanum, magnesium, manganese, nickel, osmium, platinum, rare earth metals, rhodium, ruthenium, silicon, silver, technetium, tin, titanium, vanadium, yttrium, zinc, and zirconium.

55. The medical device as defined in claim 54, wherein said metal alloy includes titanium, niobium, zirconium, and tantalum.

56. The medical device as defined in claim 54, wherein said metal alloy includes titanium, niobium, and zirconium.

57. The medical device as defined in claim 54, wherein said metal alloy includes titanium, niobium, and molybdenum.

58. The medical device as defined in claim 54, wherein said metal alloy includes chromium and molybdenum.

59. The medical device as defined in claim 54, wherein said metal alloy includes chromium, zirconium, and molybdenum.
